(19) United States
(12) Patent Application Publication
(10) Pub. No.: US 2008/0299588 A1
(43) Pub. Date: Dec. 4, 2008

(54) Methods For Measuring Bone Formation

(75) Inventor: Jeffrey Gorski, Prairie Village, KS (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: The Curators of the University of Missouri A public corporation of the State of Missouri, Kansas City, MO (US)

(21) Appl. No.: 12/130,545

(22) Filed: May 30, 2008

Related U.S. Application Data
(60) Provisional application No. 60/940,767, filed on May 30, 2007.

(51) Int. Cl. G01N 33/53

(52) U.S. Cl. 435/7.21

(57) ABSTRACT

The present invention relates to methods for detecting and monitoring bone mineralization. The invention provides antibodies, kits, and methods of use for detecting or monitoring the rate of bone mineralization associated with bone disorders such as osteoporosis.

### RELATED APPLICATIONS

This patent application claims priority from U.S. provisional patent application Ser. No. 60/940,767, filed May 30, 2007, which is incorporated herein by reference in its entirety.

### GOVERNMENTAL RIGHTS

This invention was made with government support under NIH grant number AR052775. The government has certain rights in the invention.

### FIELD OF THE INVENTION

The present invention relates to methods and kits for detecting bone mineralization. In particular, the invention relates to the detection of Bone Acidic Glycoprotein-75 (BAG-75), SKI-1, and fragments thereof.

### BACKGROUND OF THE INVENTION

Bone is a vascularized and highly dynamic tissue composed of a cellular and an extra-cellular compartment. The cellular compartment mainly consists of osteoblasts and osteoclasts, the major differentiated cells of bone. In an average person approximately 10% of the bone mass is removed and replaced each year by the continuous resorption and formation by osteoclasts and osteoblasts, respectively. The extra-cellular compartment includes the non-collagenous proteins of bone, such as bone acidic glycoprotein-75, bone sialoprotein (SP II), osteopontin, and osteonectin, which are synthesized by the cellular compartment. The acidic non-collagenous proteins of bone are important in the processes of cell recruitment and mineralization, which occur during the coupled resorptive and formative phases of bone turnover.

Alterations to the balance of bone turnover phases, caused by a wide variety of conditions, are the basis of many significant medical problems. Generally, bone mass density (BMD) is affected by reduced osteoclast activity resulting in too much density, while excess osteoclast activity results in not enough bone density. The World Health Organization (WHO) defines a BMD that is a 2.5 standard deviation (SD) or more below the average BMD for young adults as osteoporosis. One standard deviation below the norm in a measurement of hipbone density is equivalent to adding 14 years to a person's risk for fracture. Conditions associated with altered BMD include osteogenesis, osteopetrosis, Paget's disease, Rickets, and osteogenesis imperfecta. Further, bone loss and a decrease in bone mineralization is often associated with therapies used to treat non-BMD associated conditions such as HIV/AIDS, autoimmune disease, epilepsy, and juvenile rheumatoid arthritis, increasing the chances of bone fracture in the recipient.

The consequences of BMD associated medical problems include a significant impact on the financial, physical, and psychosocial well-being of the affected individual, as well as the family and community. For example, hip fractures commonly result in an inability to walk normally, and complications result in a 20 percent increase in mortality during the six months following the fracture. Nearly one-third of patients with osteoporosis associated hip fractures are discharged to nursing homes within the year following a fracture. Notably, one in five patients is no longer living one-year after sustaining the fracture. Osteoporosis, the major cause of hip fractures in older adults, can be prevented with pharmacologic agents; however, many patients who would benefit from treatment are not identified due to poor detection methods.

Currently, the most popular detection method for determining bone density is dual-energy x-ray absorptiometry (DEXA), which measures bone density throughout the body. The measurements are made by detecting the extent to which bones absorb photons that are generated by very low-level x-rays. Physicians use a formula based on the results of these procedures to determine if bone density has deteriorated to the fracture threshold set by the WHO. Unfortunately, DEXA is not widely available and may be inappropriate for many patients. Other techniques, such as ultrasound-based methods, have problems with accuracy, sensitivity, and overall predictive value. Methods that detect changes in the bone formation rate more readily than densitometric methods are needed. With earlier detection, appropriate therapies could be instituted in time to change the ultimate outcome.

Molecular markers found in biological samples are strong candidates for improved methods of detecting altered BMD. Available bone formation markers are not specifically related to the process of bone mineralization and resultantly do not have a sufficient predictive value to alterations in BMD. There is a need to develop new bone markers having specificity to bone mineralization that correlate predictably to changes in the bone mineralization rate. As new bone mineralization treatments become available, methods to identify individuals with inadequate bone repair are needed. Current methods to identify such individuals take months and have a minimum detection requirement of 1-2% loss in bone mass for detection, thus prolonging proper treatment. In this context, bone markers would allow the identification of individuals at risk of poor bone mineralization up to 5-12 months faster, thus improving the overall clinical outcome.

### SUMMARY

The inventors have discovered a method for detecting the state of bone mineralization at a specific point in time by monitoring the level of a 50 kDa fragment of Bone Acidic Glycoprotein-75 (BAG-75) or 98 kDa fragment of SKI-1, which are directly associated with the process of bone mineralization. The concentration of the 50 kDa BAG-75 fragment or 98 kDa SKI-1 fragment in a biological sample positively correlates with active bone formation. Essentially, the 50 kDa BAG-75 fragment is cleaved from the BAG-75 protein by the soluble and active 98 kDa SKI-1 fragment during the process of bone mineralization. Detection of bone mineralization may be a factor of interest in monitoring and detecting bone related diseases such as osteoporosis, osteopetrosis, Paget's disease, bone metastasis, Vitamin D deficiency, Rickets, kidney disease, hyperparathyroidism, osteogenesis imperfecta, and other conditions or treatments resulting in altered bone mineralization.

The present invention includes methods and kits relating to the detection of bone mineralization. Specifically, methods and kits utilizing BAG-75 and SKI-1 specific antibodies for the detection of the 50 kDa BAG-75 fragment, the 98 kDa SKI-1 fragment, or both in biological samples are disclosed.

The invention includes at least one antibody that recognizes at least one bone mineralization marker or combination of bone mineralization markers during the bone mineralization process. A suitable antibody of the invention may be monoclonal, polyclonal, humanized, or a fragment thereof (Fab or Fab₂). Preferably, the antibody specifically binds a peptide having at least 75%, 80%, 85%, 90%, 95%, 99% or more identity to at least 5, 8, 10, 15, 20 or more contiguous amino acids of the 50 kDa BAG-75 fragment or 98 kDa SKI-1 fragment. More preferably, the antibody specifically binds a peptide having at least 75%, 80%, 85%, 90%, 95%, 99% or more identity to at least 5, 8, 10, or 11 contiguous amino acids of the amino acid sequence of SEQ ID NO 1.

The invention includes a method of detecting bone mineralization in a biological sample using protein detection methods. The method includes providing a biological sample; assaying the sample for an altered level of at least one bone mineralization marker; and correlating the altered level with an alteration in bone mineralization. The altered level may be reduced or elevated in comparison to normal levels. Preferably, an alteration that is about a 1.5, 1.7, 2.0, 2.2, or more standard deviation above or below the normal mean is an indication there is a risk of having a BMD associated disorder.

Alternatively, the method includes providing a first biological sample; assaying the first biological sample for an altered level of at least one bone mineralization marker; providing a second biological sample; assaying the second biological sample for an altered level of at least one bone mineralization marker; comparing the amount of bone mineralization marker from each sample; and, correlating differences in the altered level of each sample with an alteration in bone mineralization. Subsequent biological samples can be provided such that the amount of at least one bone mineralization marker, is detected in a third, fourth, fifth, sixth, and seventh sample, and so forth. The detection of at least one bone mineralization marker in subsequent samples provides for a means to monitor bone mineralization over a period of time, before and after a regimen such as treatment or lifestyle change.

Suitable biological samples include blood, tissue biopsy, surgical specimen, amniotic fluid cells, sorted fetal cells from maternal circulation, autopsy material, and other body cells. Suitable protein detection methods include protein microarray analysis, enzyme linked immunosorbent assays (ELISA), Western blot, immunohistochemistry, other methods known in the art, and any combination thereof. Preferably, the bone mineralization marker may be detected by an antibody, or antibodies, that recognize at least one bone mineralization marker or a combination of bone mineralization markers. For example, the antibody may recognize the 50 kDa BAG-75 fragment exclusively or both the 50 kDa BAG-75 fragment and the BAG-75 protein.

Further, the invention includes a kit for detecting bone mineralization. The kit includes at least one antibody suitable for detecting at least one bone mineralization marker contained in a container. The supplied antibody may be labeled with detection reagents such as a radiolabel, a fluorescent tag, an enzymatic tag, a fluorogenic substrate tag, a chromogenic substrate tag, a colorimetric substrate tag, or other detectable label known in the art. Further, the antibody may be provided in a format conducive to high-throughput screening such as a multi-well plate format.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

The application contains at least one drawing executed in color. Copies of this patent application publication with color drawing(s) will be provided by the Office upon request and payment of the necessary fee. The following drawings form part of the present specification and are included to further demonstrate certain aspects of the present invention. The invention may be better understood by reference to one or more of these drawings in combination with the detailed description of specific embodiments presented herein.

FIG. 1 shows the Biomineralization Foci (BMF) that form in UMR-106-01 osteoblastic cells and can be isolated from total cell layers by laser capture microscopy (LCM). UMR-106-01 osteoblastic cells cultured in serum depleted conditions (FIG. 1A) do not exhibit altered BMF formation compared to cells cultured in the presence of serum (FIG. 1C). The cell cultures were stained with Alizarin red S to detect hydroxyapatite crystals. Only a few mineral crystals were evident in the absence of β-glycerol-phosphate (BGP) (FIG. 1B). The arrows point to mineralized BMFs (FIGS. 1A and C) and the scale bar represents 500 μm. FIGS. 1D-F show BMFs isolated by LCM of Alizarin red S stained UMR-106-01 cell culture. The arrows refer to the same BMF structure in each panel. FIG. 1D shows the microscopic field to be laser captured; FIG. 1E shows the appearance of the residual cell layer left after laser dissection; and FIG. 1F shows purified BMFs temporarily affixed to the cap used for laser capture. The scale bar represents 25 μm.

FIG. 2 demonstrates that the LCM-captured BMFs exhibit distinctive glyco- and phosphoprotein staining patterns compared with the total cell layer fraction. The asterisks emphasize the quantitative enrichment of 75-80 kDa glycophosphoproteins and of a 65 kDa Sypro ruby stained protein in the BMF lanes. The molecular weight estimates refer to blue pre-stained standards co-electrophoresed on the same gel. (KEY: buffer, extraction buffer alone; BMF, represents proteins extracted from purified BMF (FIG. 1F); +CL, cell layer extract of cultures after 24 hour (hr) mineralization in β-glycerol phosphate (FIG. 1D); −CL, cell layer extract of cultures not treated with β-glycerol phosphate (FIG. 1B)).

FIG. 3 demonstrates that BAG-75 and Bone Sialoprotein (BSP) proteins are two major glycoproteins in bone. The 75 kDa BAG-75 protein (FIG. 3A) and the 50 kDa BAG-75 fragment (FIG. 3B) were enriched in BMFs compared to the total cell layer (+CL) in the presence of BGP. Also, the 75 kDa BSP protein (FIG. 3C) and 45 kDa BSP fragments (FIGS. 3D and E) were enriched in BMFs compared to the total cell layer, in the presence of BGP. The arrows indicate 45-50 kDa fragments of BAG-75 and BSP. (KEY: BMF, extract of LCM-captured biomineralization foci; +CL, extract of total cell layer from BGP treated cultures; −CL, extract of total cell layer from cultures not treated with BGP; buffer, extraction buffer alone). BAG-75 and BSP proteins were also predominantly found in primary bone extracts. The mineralized compartment of Rat bone contained a single 75 kDa glycoprotein band reactive with Maackia amurensis agglutinin (MAA) lectin, Stains All staining, and antibodies specific for BAG-75 (FIG. 3F). Purified BSP and BAG-75 proteins exhibited a similar 75 kDa band detected with Stains All staining (FIG. 3G) to that found in FIG. 3F. Also, the 50 kDa BAG-75 fragment was revealed with Stains All staining (FIGS. 3F and G) and BAG-75 antibody detection (FIG. 3F), but not MAA lectin detection (FIG. 3H).

FIG. 4 graphically illustrates that the serine protease inhibitor AEBSF specifically inhibited mineral nucleation in both serum containing and serum-depleted conditions, while displaying higher effectiveness with mineralization competent cultures. A four-fold increase in sensitivity was observed in converting from serum-sufficient conditions to serum-depleted conditions, while a 10-fold increase in effectiveness was obtained when comparing 64-88 hour versus 44-64 hour cultures (FIG. 4A). (Key: For 64-88 hour cultures: (-□-), MTT absorbance in serum depleted conditions; (-▪-), MTT absorbance in serum containing media; (-◯-), amount of Alizarin red bound in serum-depleted conditions; (--), amount of Alizarin red bound in serum containing media. For 44-64 hour cultures: (-▾-), amount of Alizarin red bound in serum containing media). For the MTT cell viability assays, individual results (*) were significantly different from cultures treated with 1 mM AEBSF at a 99.9% confidence level. For Alizarin red S assays on 64-88 hour cultures, individual results were significantly different from cultures treated with 0.04 mM AEBSF (+) and with 0.01 mM AEBSF (#), respectively, at a 99.9% confidence level. For Alizarin red S assays on 44-64 hour cultures, individual results (**) were significantly different from cultures treated with 1 mM AEBSF at a 99.8% confidence level. FIG. 4B graphically demonstrates that AEBSF is only toxic at concentrations above 0.4 mM, regardless if cells were grown in serum-sufficient or serum-depleted conditions. (Key: (-□-), MTT absorbance in serum containing conditions; and (-◯-), amount of Alizarin red bound in serum containing conditions). MTT assay results and the amount of Alizarin red S bound to mineral deposits within cultures on day 12 are plotted versus the concentration of AEBSF added to cultures on day 9. For Alizarin red S dye binding results of primary mouse calvarial cultures, untreated controls (‡) and 0.003 mM AEBSF wells (‡) were significantly different from those treated with higher concentrations of AEBSF at a 99.6% confidence level. For MTT assay results of primary mouse calvarial cultures, untreated controls (#), 0.003 mM AEBSF wells (#), and 0.03 mM AEBSF wells (#) were significantly different from those treated with 0.01 and 0.1 mM AEBSF at a 99.4% confidence level. Results depicted are representative of three experiments. Error bars refer to the standard deviation of the mean. UMR culture studies were carried out in triplicate, while primary culture studies were carried out in quadruplicate. All analyses are based on a one-way ANOVA comparison with use of a Student-Newman-Keuls multiple comparison test.

FIG. 5 demonstrates that the enrichment of a 75 kDa phosphoglycoprotein band in the cell layers of mineralizing cultures is blocked by AEBSF. The 75 kDa glycoprotein band (arrows) detected in the cell layer and media fractions (FIG. 5A) is likely composed of BAG-75 (FIG. 5C) and BSP (FIGS. 5D and E) proteins. The 75 kDa phosphoprotein band (arrows, FIG. 5B) is presumed to be predominantly composed of BAG-75, since BSP from bone exhibits a low phosphate content while BAG-75 contains 44 phosphates/mole. Loss of the 75 kDa proteins from the media fraction only occurs when mineralization is occurring, not when it is blocked by inclusion of AEBSF or when BGP is omitted (FIGS. 5A, B, C, D, and E).

FIG. 6 demonstrates that a two-step extraction method yields increased recoveries of 75 kDa and 50 kDa glycoprotein and phosphoprotein bands. Urea-CHAPS extracts showed few differences among different BGP and AEBSF conditions (FIGS. 6A, B, and C). In contrast, EDTA extracts of cell layers grown only in the presence of BGP exhibited increased glycoprotein (FIG. 6A) and phosphoprotein (FIG. 6B) stained bands at 50 kDa and 75 kDa molecular weights when compared directly to that of cultures grown in the other conditions. Further, general protein staining with Coomassie blue yielded a comparable pattern for all culture conditions suggesting an absence of large-scale proteolysis accompanying mineral nucleation within BMFs (FIG. 6C). Compared to results with the 1-step extraction method (FIG. 5), increased recoveries of 75 kDa and 50 kDa glycoprotein and phosphoprotein bands are denoted by arrows. For reference, the appearance of relevant conditioned media gel lanes is depicted in FIG. 5A; the conditioned media was unaffected by choice of cell layer extraction method.

FIG. 7 graphically illustrates that one or more media components are required for mineralization of BMFs. Timed replacement of conditioned media establishes functionally its quantitative contribution to mineral nucleation. Mineralization was quantitated by Alizarin red S (ARS). For the ARS assay, individual results (*) were significantly different from control cultures at a 99.9% confidence level. ARS staining between control, 66 hour, and 68 hour cultures could not be distinguished statistically. (KEY: Con, control cultures whose media was replaced at 64 hours with Mineralization Media as usual; −BGP, cultures without BGP).

FIG. 8 demonstrates that mineralization occurs coincident with cleavage of BAG-75 and BSP. AEBSF inhibited proteolytic cleavage of BAG-75 and BSP as detected by BAG-75 specific antibodies (FIGS. 8A and B), BSP specific antibodies (FIG. 8C), and MAA lectin staining (FIG. 8D). The 45-50 kDa fragments of BAG-75 (FIG. 8A) and BSP (FIG. 8C) were detected in the cell layer only when mineralization occurred. Full-length BAG-75 and BSP were taken up by the cell layer only when mineralization occurred within BMFs (FIGS. 8B and C). MAA lectin, which recognizes both BSP and BAG-75, also recognizes 45-50 and 75 kDa forms in mineralized cell layer fractions (FIG. 8D). A 75 kDa glycoprotein is redistributed during mineral crystal nucleation (FIG. 8D). Immunostaining for BAG-75 and BSP proteins, and MAA lectin shows the presence of fragments in EDTA extracts from mineralizing conditions only (arrows), and the loss of full-length forms from the conditioned media in mineralizing conditions only (arrowheads).

FIG. 9 shows the identification of three additional proteins whose fragmentation is blocked by AEBSF. Cell layer EDTA extracts from cultures grown in the presence of BGP (FIG. 9A) and cultures grown in the presence of BGP with 0.04 mM AEBSF (FIG. 9B) were subjected to 2-D gel electrophoresis. Circles 1-3 represent protein spots that were excised and identified by mass spectrometry. Three AEBSF-sensitive cleavages were identified based on fragment size, pI, and differential staining properties.

FIG. 10 shows that actively mineralizing UMR 106 cells express a 98 kDa active, soluble form of SKI-1 within BMF. Specifically, the western blot in FIG. 10 shows the presence of the 98 kDa form of SKI-1 in BMF and mineralizing cultures (+CL), while it is present as smaller fragments immunoreactive fragments (<35 kDa) in un-mineralizing cultures (−CL). No bands were detected in the negative control lane (buffer).

FIG. 11 shows that the 50 kDa cleavage fragment of BAG-75 can be identified in human serum. Specifically, FIG. 11 depicts a western blot showing that normal human serum contains an approximately 50 kDa band that reacts similarly with anti-VARYQNTEEEE antibodies as does that for freshly prepared ovariectomized rat serum (OVX) and sham-operated rat serum (sham). The 50 kDa protein content after ovariectomization was higher than in sham-operated rat serum (small arrow, rat 50 kDa). The human 50 kDa band (small arrow, human 50 kDa) is slightly larger than that for rat due to difference in cleavage or phosphorylation. The position of the human serum albumin non-reactive negative band is noted with a large arrow.

### DETAILED DESCRIPTION

It has been discovered that cleavage of BAG-75 is a critical determinant of bone mineralization. Specifically, inhibiting serine protease fragmentation or cleavage of BAG-75 prevents bone mineralization. As a consequence, the 50 kDa cleavage fragment of BAG-75 and SKI-1 serine protease are promising candidates for bone mineralization markers for monitoring and detecting the rate of bone mineralization using biological samples. The present invention includes methods and kits for the detection of bone mineralization, as well as methods that utilize antibodies directed to BAG-75, the 50 kDa BAG-75 cleavage fragment, and SKI-1 protease.

### I. Methods of the Invention

A. Methods

The invention includes a method for detecting or monitoring bone mineralization using bone mineralization markers found in biological samples. The term “bone mineralization marker” as used herein includes BAG-75, the 50 kDa fragment of BAG-75, fragments of BAG-75, SKI-1, fragments of SKI-1, BSP, and fragments of BSP. The method includes providing a biological sample, assaying the provided sample for at least one bone mineralization marker or any combination of bone mineralization markers, and correlating altered levels of the bone mineralization marker or combination of bone mineralization markers to an alteration in bone mineralization. Methods of obtaining a biological sample are well known in the art and may include drawing blood or a spinal tap. Methods for assaying a protein in a biological sample are known in the art and methods for specifically assaying levels of bone mineralization markers are described herein and in U.S. Pat. No. 5,637,446 and incorporated herein by reference. A skilled artisan will recognized that the methods known in the art can be easily altered for the assaying of any one of the bone mineralization markers.

The level of bone mineralization marker or combinations may be reduced or elevated compared to normal levels of the bone mineralization marker or combination. Normal levels are established by assaying a variety of biological samples, such as serum or synovial fluid, from age and gender matched subjects for the level of bone mineralization marker. Preferably, the subjects are not genetically predisposed to a BMD associated disorder and have normal BMD levels according to currently available screening methods, such as DEXA. Methods of detecting bone mineralization markers in a biological sample are described herein and in U.S. Pat. No. 5,637,466, incorporated herein by reference. A skilled artisan will recognize that a sufficient sample size is necessary to establish a normal level or range of bone mineralization markers found in a biological sample for a given sample population.

The normal level or range is dependent upon numerous variables including age, gender, weight, activity, diet, and environment. As such, test sample levels should be compared to ranges established in an appropriate comparison population. Suitable comparison population ranges are those that are age and gender matched to the test sample. For example, the test sample from a thirty-year-old female subject is compared to a range established using a sample population of females about thirty years old. Likewise, the test sample from a sixty-year-old male subject is compared to a range established using a sample population of males about sixty years old.

Altered levels of bone mineralization markers with an increased or decreased level, in comparison to the established normal range of an appropriate comparison population, indicate a risk for BMD associated disorders. For example, altered levels of bone mineralization markers that are at least about a 1.5 standard deviation below or above the established normal range are at risk of having a BMD associated disorder. For a given population, there is a range inclusive of the level of bone mineralization marker for each sample in the population. Some resultant levels are above the calculated mean of the population, some are the same as the mean, and some are below the mean. The range above the mean and the range below the mean are divided into thirds, and each third is referred to as a standard deviation. There are six standard deviations, including three above the mean and three in the range below the mean. Altered levels of bone mineralization markers that are at least about a 1.5 standard deviation above or below the mean of the appropriate comparison population are at risk of having a BMD associated disorder. Altered levels of bone mineralization markers that are at least about a 1.6, 1.8, 2.0, or 2.2 standard deviation above or below the mean of the appropriate comparison population are at an increased risk of having a BMD associated disorder. Altered levels of bone mineralization markers that are at least about a 2.3, 2.5, 2.7, 3.0, or more standard deviation above or below the mean of the appropriate comparison population have a BMD associated disorder.

Another method of the invention includes providing multiple biological samples, assaying each for altered levels of bone mineralization markers, correlating altered levels with altered bone mineralization. The level of bone mineralization marker detected may be compared to the levels of the other samples and an alteration between the samples can be correlated to altered bone mineralization. The multiple samples may be collected over a period of time or before and after a regimen, such as a therapeutic treatment or lifestyle change, to detect or monitor bone mineralization over time or in response to a regimen. The number and frequency of biological samples collected may be 1, 2, 3, 4, 5, 6, 7, 8, 9, 10, or more over a period of a day, a week, a month, several months, a year, or more. A skilled artisan will recognize and appreciate that the number and frequency depends upon the intended use of the method.

Exemplary biological samples include bodily fluids such as peripheral blood or serum, synovial fluid, saliva, a tissue biopsy, surgical specimen, amniotic fluid, and autopsy material. Proteins can be isolated by methods commonly known in the art and described in Current Protocols in Protein Science, Unit 4, pub. John Wiley & Sons, Inc., 2004 and incorporated herein by reference.

As used herein, the phrase “protein detection method” refers to methods commonly used in the art to detect specific proteins in a sample. Suitable protein detection methods include those that can detect bone mineralization markers in a sample. Methods of detecting proteins in a biological sample are commonly known in the art. Exemplary methods include protein microarray analysis, enzyme-linked immunosorbent assays (ELISA), Western blot, immunohistochemistry, and other methods known in the art. Preferably, the invention includes methods of using at least one antibody directed to at least one bone mineralization marker to detect bone mineralization. For example, proteins extracted from a biological sample by methods known in the art may be run on a denaturing protein gel. The separated proteins are then transferred to a membrane that is probed with a BAG-75 specific antibody and a horseradish peroxide linked secondary antibody. Presence of BAG-75 or fragments thereof, in a sample will be recognizable by chemiluminescent detection of the antibodies. Also, more than one antibody directed to a combination of bone mineralization markers may be used to detect bone mineralization. By way of example, proteins extracted from a biological sample may be separated in a denaturing protein gel and transferred to a membrane. The membrane may be probed with a BAG-75 specific antibody and a SKI-1 specific antibody followed by horseradish peroxide linked secondary antibodies. An increased level of the 50 kDa fragment of BAG-75 or 98 kDa fragment of SKI-1 above the normal standard, indicates active bone mineralization.

B. Subjects

Methods of the invention may be used to identify or monitor particular subjects with, or at risk of bone mineralization defects. Subjects with, or at risk of, bone mineralization defects, include but are not limited to the following: malnourished subjects; subjects living in poverty or malnutrition conditions; subjects that are elderly or chronically ill; subjects with bone disease such as those with osteoporosis, Paget's disease, bone metastasis, Rickets, osteogenesis imperfecta or other bone disease associated with altered bone mineralization; subjects with autoimmune diseases, kidney disease, hyperparathyroidism, or Vitamin D deficiency; subjects being treated with treatments resulting in bone loss such as those for HIV/AIDS, autoimmune disease, epilepsy, juvenile rheumatoid arthritis, chronic glucocorticoid therapy and the like; and subjects undergoing bone repair or healing. A skilled artisan will recognize that the methods of the invention may appropriately be used to monitor or detect other diseases and disorders associated with or resulting from defective bone mineralization.

The methods of the present invention may be utilized for any mammalian subject. Such mammalian subjects include, but are not limited to, human subjects or patients. The methods are particularly useful in screening subjects to diagnose or monitor osteoporosis and other bone mineralization defect associated disorders. Exemplary subjects may also include domesticated mammals (e.g., dogs, cats, horses), mammals with significant commercial value (e.g., dairy cows, beef cattle, sporting animals), mammals with significant scientific value (e.g., captive or free specimens of endangered species), or mammals which otherwise have value.

### II. Compositions

A. Antibodies

The present invention provides for antibodies and antibody fragments that bind to bone mineralization markers. Preferably, the antibodies bind to at least one of the following: BAG-75, 50 kDa cleavage product of BAG-75, SKI-1, or the 98 kDa fragment of SKI-1. The antibodies of the invention include those that distinctly bind and recognize the fragments of BAG-75, specifically the 50 kDa BAG-75 fragment. Specifically, the invention includes antibodies that recognize the BAG-75 amino acid sequence VARYQNTEEEE of SEQ ID NO 1 (U.S. Pat. No. 5,637,466).

Antibodies of the invention may be of any type known in the art including, but not limited to, polyclonal, monospecific polyclonal, monoclonal (mAbs), recombinant, chimeric, humanized such as CDR-grafted, human, single chain, and bispecific, as well as fragments, variants or derivatives thereof. Antibody fragments include those portions of the antibody that bind to an epitope on the BAG-75 peptide or the SKI-1 peptide. Exemplary fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions. Methods for making antibodies specific for BAG-75 peptides or SKI-1 peptides are commonly known in the art and described in Brigstock et al., J. Biol. Chem., 275: 24953-61, 1997 and Antibodies: A Laboratory Manual, Cold Spring Harbor Laboratory, 1988; both incorporated herein by reference.

1. Antigens for Antibody Production

Suitable amounts of well-characterized antigen for production of antibodies can be obtained using standard techniques known in the art such as, but not limited to, cloning or synthetic synthesis. Antigenic proteins can be obtained from transfected cultured cells that overproduce the antigen of interest. For example, expression vectors that have nucleotide sequences encoding an antigen of interest can be constructed, transfected into cultured cells, and then the antigen can be subsequently isolated using methods well-known to those skilled in the art (see, Wilson et al., J. Exp. Med. 173:137, 1991; Wilson et al., J. Immunol. 150:5013, 1993). Alternatively, DNA molecules encoding an antigen of choice can be obtained by synthesizing DNA molecules using mutually priming long oligonucleotides (see, Ausubel et al., (eds.), Current Protocols In Molecular Biology, pages 8.2.8 to 8.2.13, 1990; Wosnick et al., Gene 60:115, 1987; and Ausubel et al. (eds.), Short Protocols In Molecular Biology, 3rd Edition, pages 8-8 to 8-9, John Wiley & Sons, Inc., 1995). As a skilled artisan will recognize, established techniques using the polymerase chain reaction provide the ability to synthesize antigens (Adang et al., Plant Molec. Biol. 21:1131, 1993; Bambot et al., PCR Methods and Applications 2:266, 1993; Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263 268, Humana Press, Inc. 1993). Once produced, the antigen of choice is used to generate antigen specific antibodies.

2. Antibody Production

The present invention provides antibodies as detection agents of BAG-75. It is envisioned that such antibodies include, but are not limited to, polyclonal, monoclonal, humanized, part human, or fragments thereof. A skilled artisan will appreciate the benefits and disadvantages of the type of antibody used for therapeutic treatment and will further recognize the selection is dependent upon the intended use.

i. Polyclonal Antibodies

Means for preparing and characterizing polyclonal antibodies are well known to those skilled in the art (see, e.g., Antibodies: A Laboratory Manual, Cold Spring Harbor Laboratory, 1988; incorporated herein by reference). For example, for the preparation of polyclonal antibodies, the first step is immunization of the host animal with the target antigen, where the target antigen will preferably be in substantially pure form, with less than about 1% contaminant. The antigen may include the complete target protein, fragments, or derivatives thereof. To prepare polyclonal antisera an animal is immunized with an antigen of interest, and antisera is collected from that immunized animal. A wide range of animal species can be used for the production of antisera. Typically the animal used for production of anti-antisera is a rabbit, mouse, rat, hamster, guinea pig or goat. Because of the relatively large blood volume of rabbits, a rabbit is a preferred choice for the production of polyclonal antibodies.

The amount of antigen used in the production of polyclonal antibodies varies upon the nature of the antigen as well as the animal used for immunization. A variety of routes can be used to administer the antigen of choice; subcutaneous, intramuscular, intradermal, intravenous, intraperitoneal and intrasplenic. The production of polyclonal antibodies may be monitored by sampling blood of the immunized animal at various points following immunization. A second, booster injection, may also be given. The process of boosting and titering is repeated until a suitable titer is achieved. When a desired titer level is obtained, the immunized animal can be bled and the serum isolated and stored. The animal can also be used to generate monoclonal antibodies, as is well known to those skilled in the art.

The immunogenicity of a particular composition can be enhanced by the use of non-specific stimulators of the immune response, known as adjuvants. Exemplary adjuvants include complete Freund's adjuvant, a non-specific stimulator of the immune response containing killed Mycobacterium tuberculosis; incomplete Freund's adjuvant; and aluminum hydroxide adjuvant.

It may also be desired to boost the host immune system, as may be achieved by associating the antigen with, or coupling the antigen to, a carrier. Exemplary carriers include keyhole limpet hemocyanin (KLH) and bovine serum albumin (BSA). Other albumins such as ovalbumin, mouse serum albumin or rabbit serum albumin can also be used as carriers. As is also known in the art, a given composition may vary in its immunogenicity.

ii. Monoclonal Antibodies

Monoclonal antibodies (Mabs) may be readily prepared through use of well-known techniques to those skilled in the art, such as those exemplified in U.S. Pat. No. 4,196,265, incorporated herein by reference. Typically, this technique involves immunizing a suitable animal with the selected antigen. The antigen is administered in a manner effective to stimulate antibody-producing cells. Rodents such as mice and rats are preferred animals, however, the use of rabbit, sheep and frog cells is also possible.

By way of example, following immunization, the somatic cells with the potential for producing antigen specific antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The anti-antigen antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas).

Any one of a number of myeloma cells may be used, as are known to those of skill in the art (Goding, pp. 65 66, 1986; Campbell, pp. 75 83, 1984; each incorporated herein by reference). For example, where the immunized animal is a mouse, one may use P3-X63/Ag8, X63-Ag8.653, NS1/1.Ag 41, Sp210-Ag14, FO, NSO/U, MPC-11, MPC11-X45-GTG 1.7 and S194/5XX0 Bul; for rats, one may use R210.RCY3, Y3-Ag 1.2.3, IR983F, 4B210 or one of the above listed mouse cell lines; and U-266, GM1500-GRG2, LICR-LON-HMy2 and UC729-6, are all useful in connection with human cell fusions.

The heterogeneous cell population may be cultured in the presence of a selection medium to select out the hybridoma cells. A suitable selection medium includes an inhibitor of de novo synthesis, such as aminopterin in HAT medium, methotrexate in HMT medium, or azaserine in AzaH medium plus the necessary purine and pyrimidine salvage precursors (i.e. hypoxanthine and thymidine in HAT or HMT media; hypoxanthine in AzaH medium). Only cells capable of operating nucleotide salvage pathways are able to survive in the selection medium. The myeloma cells are defective in key enzymes of the salvage pathway, e.g., hypoxanthine phosphoribosyl transferase (HPRT), and cannot survive. The B cells can operate this pathway, but they have a limited life span in culture and generally die within about two weeks. Therefore, the only cells that can survive in the selective media are those hybrids formed from myeloma and B cells (hybridomas).

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired anti-antigen reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual anti-antigen antibody-producing cell lines, which clones can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations.

MAbs produced by either means will generally be further purified, e.g., using filtration, centrifugation and various chromatographic methods, such as HPLC or affinity chromatography, all of which purification techniques are well known to those of skill in the art. These purification techniques each involve fractionation to separate the desired antibody from other components of a mixture. Analytical methods particularly suited to the preparation of antibodies include, for example, protein A-Sepharose and protein G-Sepharose chromatography.

iii. Humanized Antibodies

Also of interest are humanized antibodies. Methods of humanizing antibodies are known in the art. The humanized antibody may be the product of an animal having transgenic human immunoglobulin constant region genes (see for example International Patent Applications WO 90/10077 and WO 90/04036, both incorporated herein by reference). Alternatively, the antibody of interest may be engineered by recombinant DNA techniques to substitute the CH1, CH2, CH3, hinge domains, and the framework domain with the corresponding human sequence (WO 92/02190 and incorporated herein by reference).

The use of Ig cDNA for construction of chimeric immunoglobulin genes is known in the art (Liu et al. P.N.A.S. 84:3439, 1987 and incorporated herein by reference). mRNA is isolated from a hybridoma or other cell producing the antibody and used to produce cDNA. The cDNA of interest may be amplified by the polymerase chain reaction using specific primers (see U.S. Pat. Nos. 4,683,195 and 4,683,202, both incorporated herein by reference). Alternatively, a library is made and screened to isolate the sequence of interest. The DNA sequence encoding the variable region of the antibody is then fused to human constant region sequences. The sequences of human constant region genes may be found in Kabat et al. Sequences of Proteins of Immunological Interest, N.I.H. publication no. 91-3242, 1991 and incorporated herein by reference. Human C region genes are readily available from known clones. The chimeric, humanized antibody is then expressed by conventional methods known to those of skill in the art.

iv. Antibody Fragments

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule. The following patents and patent applications are specifically incorporated herein by reference for the preparation and use of functional, antigen-binding regions of antibodies, including scFv, Fv, Fab′, Fab and F(ab′)₂ fragments: U.S. Pat. Nos. 5,855,866; 5,965,132; 6,051,230; 6,004,555; and 5,877,289.

Also contemplated are diabodies, which are small antibody fragments with two antigen-binding sites. The fragments may include a heavy chain variable domain (V_{H}) connected to a light chain variable domain (V_{L}) in the same polypeptide chain (V_{H} V_{L}). By using a linker that is too short to allow pairing between the two domains on the same chain, the domains are forced to pair with the complementary domains of another chain and create two antigen-binding sites. Techniques for generating diabodies are well known to those of skill in the art and are also described in EP 404,097 and WO 93/11161, each specifically incorporated herein by reference. Also, linear antibodies, which can be bispecific or monospecific, may include a pair of tandem Fd segments (V_{H} C_{H1}-V_{H} C_{H1}) that form a pair of antigen binding regions may be useful to the invention as described in Zapata et al. (1995), and incorporated herein by reference.

Antibodies that specifically bind to BAG-75 or SKI-1 may be used in diagnostic assays for the detection of the BAG-75 or SKI-1 polypeptides in various body fluids. In another embodiment, the BAG-75 peptide or SKI-1 peptide may be used as antigens in immunoassays for the detection of BAG-75 in various patient tissues and body fluids including, but not limited to: ambiotic fluid, blood, serum, ear fluid, spinal fluid, sputum, urine, lymphatic fluid and cerebrospinal fluid. The antigens of the present invention may be used in any immunoassay system known in the art including, but not limited to: radioimmunoassays, ELISA assays, sandwich assays, precipitin reactions, gel diffusion precipitin reactions, immunodiffusion assays, agglutination assays, fluorescent immunoassays, protein A immunoassays, and immunoelectrophoresis assays.

For diagnostic applications, antibodies that specifically bind BAG-75 or SKI-1 may be labeled with a detectable moiety. A suitable detectable moiety includes those known in the art that are capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase, or horseradish peroxidase (Bayer et al., Meth. Enz., 184:138-163, 1990).

### III. Kits

The invention includes at least one kit suitable for assaying for the presence of BAG-75 or SKI-1 in a biological sample. The kit includes reagents necessary for the detection of BAG-75 or a fragment thereof, including the 50 kDa cleavage product of BAG-75. The kit may also include reagents necessary for the detection of SKI-1 or fragment thereof, including the 98 kDa soluble form of SKI-1. Exemplary reagents may include a BAG-75-specific antibody, a 50 kDa BAG-75 fragment-specific antibody, detection reagents, and other reagents useful in the art for detecting the presence of a specific protein in a biological sample. Further, reagents may include a SKI-1-specific antibody or a 98 kDa SKI-1 fragment-specific antibody. Antibodies may be tagged with a radiolabel, a fluorescent tag, an enzymatic tag, a fluorogenic substrate tag, a chromogenic substrate tag or other tag known in the art for detection purposes. Likewise, suitable detection reagents may include secondary antibodies to detect non-tagged primary antibodies. A secondary antibody may be tagged with a radiolabel, a fluorescent tag, an enzymatic tag, a fluorogenic substrate tag, a chromogenic substrate tag or other tag known in the art. Further, the kit may include reagents for the isolation of protein from biological samples. Suitable reagents are commonly known in the art, but may be provided for ease of kit use. The reagents may be provided in a high-throughput format such as in a micro-well plate. The kit of the invention may further include other materials desirable from a commercial and user standpoint, including buffers, diluents, filters, needles, syringes, and package inserts with instructions for use.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al, Nature 341:544-546 (1989); and Vaughan et al., Nature Biotech. 14:309-314 (1996), or by immunizing an animal with the antigen or with DNA encoding the antigen.

As used herein the term “isolated” is meant to describe a polynucleotide, a nucleic acid, a protein, a polypeptide, an antibody, or a host cell that is in an environment different from that in which the polynucleotide, nucleic acid, protein, polypeptide, antibody, or host cell naturally occurs. In reference to a sequence, such as nucleic acid or amino acid, “isolated” includes sequences that are assembled, synthesized, amplified, or otherwise engineered by methods known in the art.

The phrase “specifically binds” when referring to a protein or peptide, refers to a binding reaction that is determinative of the presence of the protein, in a heterogeneous population of proteins and other biologics. Thus, under designated immunoassay conditions, the specified antibodies bind to a particular protein sequence at least two times the background and more typically more than 10 to 100 times background. Specific recognition by an antibody under such conditions requires an antibody that is selected for its specificity for a particular protein. For example, antibodies raised against a particular protein, polymorphic variants, alleles, orthologs, and conservatively modified variants, or splice variants, or portions thereof, can be selected to obtain only those polyclonal antibodies that are specifically immunoreactive with BAG-75 like peptides or fragments and not with other random proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

As used herein, the term “bone mineralization marker” includes BAG-75, fragments of BAG-75, the 50 kDa fragment of BAG-75, SKI-1, fragments of SKI-1, the 98 kDa soluble fragment of SKI-1, BSP, and fragments of BSP.

### EXAMPLES

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of non-critical parameters that could be changed or modified to yield essentially similar results.

### Example 1

### Materials and Methods

The following materials and methods are used throughout the examples.

Cell Culture. UMR 106-01 cells were maintained and cultured at 37° C. and 5% carbon dioxide. Cells were seeded at a density of 1.0×105 cells/cm² in Growth Medium (Eagle's MEM supplemented with Earle's salts, 1% non-essential amino acids, 10 mM, HEPES, pH 7.2, and 10% fetal bovine serum). After 24 hours (h), the medium was exchanged with Growth Medium containing 0.5% BSA. Sixty-four hours after plating, the culture medium was exchanged with Mineralization Media (Growth Medium containing either 0.1% BSA or 10% fetal bovine serum and 7 mM β-glycerol phosphate). Cultures were then incubated for an additional 24 hours, at the end of which (88 h), the cells were fixed in 70% ethanol and either subjected to MTT assay or extracted for protein. In some experiments, protease inhibitors, including serine protease inhibitor AEBSF (4-(2-aminoethyl)-benzenesulfonylfluoride HCl), were added to cultures at 64 h after plating in Mineralization Media. Alternatively, AEBSF was added at 44 h after plating; inhibitor was then removed and exchanged for Mineralization Media at 64 h and the amount of mineralization analyzed at 88 h.

Primary mouse calvaria were isolated from 3-5 day old mice and digested with trypsin/collagenase. Three sequential enzymatic digestions were pooled and spun down at 1500 rpm and the cells were resuspended in alpha-MEM containing 10% fetal bovine serum, 2 mM L-glutamine, 100 u/ml penicillin and 30 ug/ml gentamicin (Alpha-Growth Medium). Cells were plated directly at a density of 2-3×106 cells per T-75 cm2 flask and allowed to reach confluency (3-4 days). Confluent cultures were passaged by trypsinization and re-plating in 12- or 24-well culture dishes at a density of 20,000 cells per cm2. Cultures were subsequently re-fed at three-day intervals with Alpha-Growth Medium supplemented with 50 ug/ml ascorbic acid and 5 mM beta-glycerol phosphate. Beta-glycerol phosphate was omitted from some wells, which served as an unmineralized control. In order to test the effect of AEBSF, separate duplicate cultures were treated on days 3, 6, or 9 with Alpha-Growth Medium supplemented with 50 ug/ml ascorbic acid and 5 mM beta-glycerol phosphate containing 0.003 mM to 0.1 mM AEBSF. Phase contrast images were taken of living cultures on days 3-12. On day 12 after plating, one set was incubated with MTT as described for UMR 106. A second set was fixed on day 12 with 70% ethanol and processed for quantitative Alizarin red S staining as described for UMR cultures.

MTT Assays Culture wells were washed with Eagle's MEM supplemented with Earle's salts and then incubated with a solution of 0.5 mg/ml MTT (3-(4,5-dimethylthiazol-2-yl)-2,5-diphenyl-2Htetrazolium bromide) in Eagle's MEM for 1-2 h at 37° C. Excess MTT solution was removed, the cells disrupted by mixing briefly with dimethylsulfoxide, and free, reduced dye was read at 490 or 540 nm in a spectrophotometer.

Quantitation of Mineralization. After fixation in 70% ethanol, the cell layer was rinsed with a 2:1 solution of 70% ethanol/TBS (Tris-acetate buffer (pH 7.5) containing 0.15 M sodium chloride) for 2 minutes. The cell layer was stained with 4 mM Alizarin red S for 5 minutes. To remove background staining, wells were washed briefly 7 times in deionized water, once in TBS, once in deionized water, and finally with PBS. Bound dye was quantitated by extraction for 2 h at 4° C. in cold 10 mM HCl in 70% ethanol. The initial extract was collected and combined with a second extract and then read at 520 nm in a spectrophotometer. A standard curve for Alizarin red S dye was constructed for each analysis and the amount of bound dye/culture well was determined. A separate, but related protocol was used with serum-depleted cultures. After fixation in 70% ethanol, the cell layer was rinsed one time with 1 mM HEPES and then stained with 4 mM Alizarin red S for 10 minutes at room temperature while rotating slowly. After staining, the cell layer was then rinsed with 1 mM HEPES in nanopure water. Bound dye was then extracted for 15 minutes at 4° C. in cold 10 mM HCl in 70% ethanol. The initial extract was collected and combined with a second repeat extraction. Combined extracts were read at 520 nm in a spectrophotometer and the amount of bound dye/culture well was determined based on a standard curve.

Statistical Methods. All statistical tests were performed using SigmaStat 3.1 software. A one-way analysis of variance (ANOVA) test was used to determine if a statistical difference existed between the viability of UMR-106-01 cultures or the amount of mineral deposited. Subsequent pair-wise multiple comparison tests were performed with the Student-Newman-Keuls method or the Kiruskal-Wallis method as noted.

Extraction of Cell Layer Fraction—1-Step Method. Cell were dislodged by scraping and then extracted with 75 mM potassium phosphate buffer (pH 7.2), containing 10 mM CHAPS, 75 mM sodium chloride, 50 mM tetrasodium EDTA, 10 mM benzamidine hydrochloride, 2 mM DTT and 0.02% sodium azide for 1 h at 4° C. Each extract was then homogenized briefly using a motorized pestle and then clarified by ultracentrifugation at 30,000 rpm for 1 h at 4° C. in a SW 50.1 rotor prior to use. Conditioned media was immediately heated at 95° C. for 5 minutes to inactivate protease activity and then frozen at −80° C. until analyzed.

Two Step Extraction of Cell Layer—2-Step Method. During the final 24 hour mineralization period, cells were grown in BSA-free, serum-free media conditions to reduce the amount of BSA in fractions used for 2-dimensional gel electrophoresis. Media was removed from each flask, heated at 95° C. for 5 minutes, dialyzed against 5% acetic acid, and lyophilized to dryness. Cell layers were first extracted without mixing for 2 h at 4° C. in 0.05 M Tris-acetate buffer (pH 7.5) containing 0.15 M NaCl, 0.05 M EDTA and 0.02% sodium azide; extracts were then inactivated at 95° C. for 5 minutes, dialyzed against 5% HAc, and lyophilized to dryness. The residual cell layer was next dislodged by scrapping and then extracted overnight at 4° C. by slow mixing with 0.1 M Trisacetate buffer (pH 7.5) containing 8 M urea, 2% (w/v) CHAPS and 0.02% sodium azide. Urea extracts were then homogenized and clarified by ultracentrifugation at 30,000 rpm for 1 h at 4° C. in an SW 50.1 rotor prior to use in 2-dimensional gel electrophoresis.

Western Blotting—Chemiluminescence Detection. Cell layer extracts and media fractions prepared as described above were electrophoresed on 4-20% linear gradient gels and electroblotted onto PVDF membranes for 2 h at 100 volts. The transfer buffer was composed of 10 mM CAPS buffer (pH 11.0) containing 10% methanol. After transfer, membranes were blocked for 1 h in 5% nonfat dry milk in 1× TBST. Blots were then rinsed for 15 minutes in 1× TBST followed by three additional washes for 5 minutes each. Primary antibodies were diluted in blocking solution and the blots incubated overnight in diluted primary. Blots were then washed in 1× TBST and then incubated in the dark for 2 h in either secondary horseradish peroxidaseconjugated anti-mouse or anti-rabbit heavy and light chain IgG antibody. Blots were again washed in 1× TBST and then exposed to chemiluminescence reagents for 5 minutes prior to exposure to x-ray film; films were digitized using a flat bed scanner.

Laser Capture Microscopy. UMR cells were grown as usual on Fisher Plus microscope slides, fixed, and stained with Alizarin red S dye. Immediately prior to laser capture, slides were dehydrated through a graded series of ethanol washes and xylene rinses. Dried slides were stored at −20° C. in a sealed box with desiccant until used. Mineralized BMF were collected onto standard caps using an Arturus Pixel Ile microscope. Collection films were pooled and stored in 70% ethanol at −20° C. until approximately 6200 BMF were collected. LCM-captured BMF then were mixed in 70% ethanol to dislodge the purple-stained particles which were then microfuged to remove the ethanol. BMF pellets were extracted twice sequentially over a two day period at 4° C. with 1.1 ml of 0.1 M Tris Trisacetate buffer (pH 7.8) containing with 0.5% octyl-glucoside, 0.05% SDS, 0.05 M EDTA, and 0.02% sodium azide. Extracts were then dialyzed first against 0.01 M Tris-acetate buffer (pH 7.8) containing 8 M urea, 0.05% SDS, 0.1% octyl-glucoside, 0.05 M EDTA, and second against 0.01 M Tris-acetate buffer (pH 7.8) containing 8 M urea, 0.05% SDS, and 0.1% octyl-glucoside. Controls represented glass slides containing the total cell layer fractions from +BGP or −BGP cultures; control slides were extracted using a similar protocol. The resultant dialyzed extracts were used for comparative blotting studies where identical protein amounts were loaded per gel lane.

Protein determination. Protein concentration of BMF extracts was determined using the Non-Interfering Protein Assay by Geno-Technology Inc (St. Louis, Mo.).

Mass Spectrometric Analyses. Protein bands were detected by staining with Coomassie blue G or with Sypro Ruby dye according to the manufacturer's instructions (Bio-Rad, Inc.). Excised 1-D and 2-D SDS PAGE gel bands/spots were reduced and alkylated, followed by digestion with trypsin for 6-16 h. Peptides were extracted and subjected to reversed phase capillary LC-MS with a linear 2-70% acetonitrile gradient over 45 minutes in 50 mM acetic acid (aqueous phase) using a 50 μM i.d. (inner diameter) picofrit (New Objective) capillary column packed to 8 cm with Phenomenex Jupiter Proteo C18 matrix, eluting directly into an LTQ linear ion trap mass spectrometer. The instrument was operated in the data dependent mode with one mass spectrum and eight collision induced dissociation spectra acquired per cycle. The data were analyzed using Mascot (Matrix Science, LTD), to find protein matches in the MSDB_{—}20050227.fasta database. All proteins reported in Table 2 obtained at least two different peptide matches with scores exceeding the threshold value for 95% confidence, with manual assessment of each MS/MS match to ensure validity.

2-D Polyacrylamide Gel Electrophoresis. Gels were run and stained with either colloidal Coomassie blue G, Pro-Q Emerald 300 glycoprotein stain (Invitrogen Corp.), or Pro-Q Diamond phosphoprotein stain (Invitrogen Corp.). PD-Quest (Bio-Rad Laboratories, Inc.) software was used to digitally analyze the colloidal Coomassie blue G stained gels comparing AEBSF treated with non-treated cell layer and media fractions in order to identify proteins differentially expressed in one condition versus another.

### Example 2

### Mineralization of UMR Osteoblastic Cells Unchanged in Serum-Depleted Conditions

Bone mineralization is commonly studied using cell culture. Specifically, Rat calvaria cells, including UMR 106 osteoblastic cells that form biomineralization foci (BMF), are used. Generally, the media used to grow and maintain such cells contains fetal bovine serum, which is not well defined and contains a plethora of proteins that may complicate the interpretation of experimental results. To determine if the formation of BMF in UMR cells is dependent on proteins found in fetal bovine, the affect of using serum-free conditions on the amount or morphology of mineralization in UMR 106 cultures was analyzed.

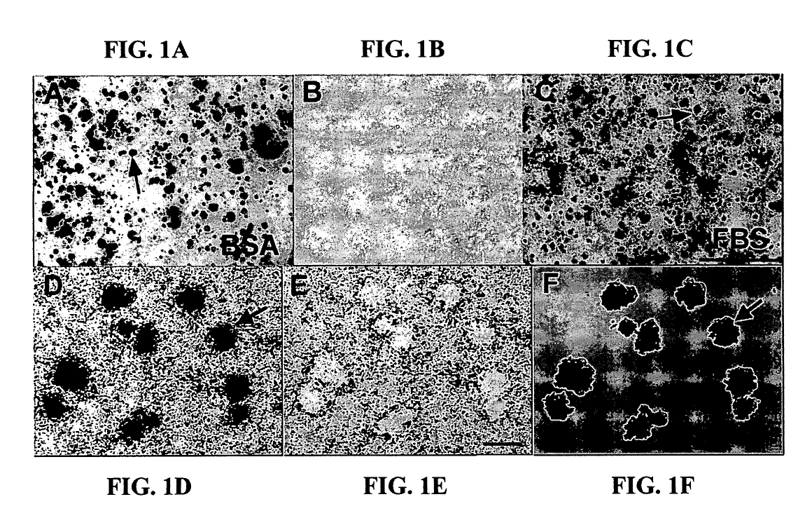

UMR 106 cultures were initially plated in 10% serum and then re-fed 16 hours later with 0.5% BSA-supplemented media. Approximately 64 hours after plating, the cultures were fed again with 0.1% BSA-supplemented media in the presence or absence β-glycerol phosphate. Cells were then fixed with 70% ethanol and stained with 4 mM Alizarin red S dye (FIGS. 1A and C). Few mineral crystals were evident when β-glycerol phosphate was omitted (FIG. 1B). Further, no differences were noted in the amount or morphology of mineralized BMF between serum-containing and serum-depleted conditions (compare FIG. 1A vs. 1C). Quantitation of the amount of Alizarin red stain bound per well also revealed no significant differences. Manual counts of mineralized BMF formed under serum-containing and serum-depleted conditions showed no statistical difference (103 foci/cm2±6.56 S.D. vs.105 mineralized foci/cm2±6.08 S.D., p=0.486 using one-way ANOVA followed by Kiruskal-Wallis method).

The presence or absence of serum had essentially no effect on the formation of BMF. The above-described results confirm that the mineralization potential is unchanged in conditions of serum depletion.

### Example 3

### BAG-75, BSP, and Fragments of Each are Enriched Within Purified Mineralized BMF

There are few bone markers that can be used for the detection of the bone mineralization process. Yet, bone mineralization is a distinct process that is likely associated with a unique proteome. The formation of BMF in Rat UMR 106 ostoblastic cells were analyzed to determine the unique proteome associated with bone mineralization and to define bone markers specific for this process.

Mineralized BMF, which appear as dark spots about 20-25 micron in diameter, were isolated from ethanol-fixed, Alizarin red stained UMR 106 cultures by laser capture microsdissection (FIGS. 1E, F, and G). Alizarin red staining was used to identify BMF structures in the initial mineral crystal nucleation stage. Laser capture microdissection allowed the isolation of mineralized BMF from the remaining culture as evidenced by the residual “holes” devoid of cells depicted in FIG. 1E. In the isolated BMF preparation, the captured BMF (FIG. 1F) remained in relatively the same orientation as they were in culture (FIG. 1D). Visual inspection of the captured populations revealed an absence of obvious cellular contamination.

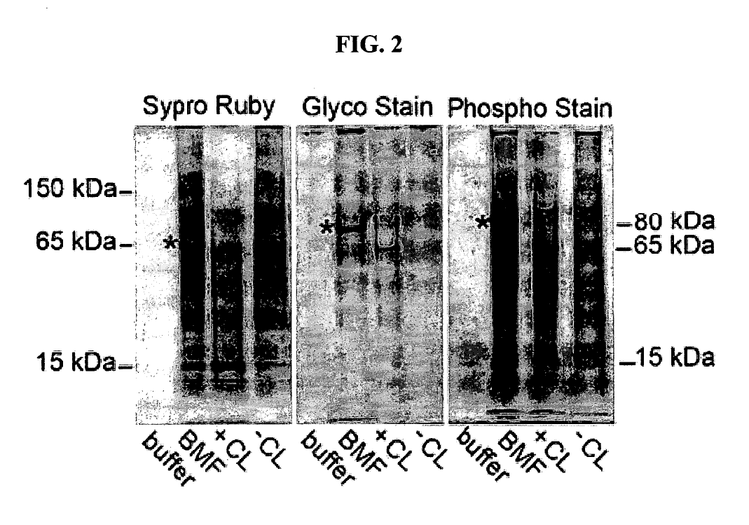

Following laser capture microdissection, proteins were extracted from approximately 6200 pooled BMF with 0.1 M Tris-acetate buffer (pH 7.8), containing with 0.5% octyl-glucoside, 0.05% SDS, 0.05 M EDTA, and 0.02% sodium azide, and then subjected to SDS PAGE. As a comparative control, UMR cultures containing the total cell layer along with mineralized BMF (FIG. 1D) were processed similarly (+CL). Cultures not treated with beta-glycerol phosphate and not containing mineralized BMF represent a second control (−CL). Equal amounts of protein were applied to each lane and gels were stained to visualize the complete proteome present (Sypro Ruby), glycoproteins (Glyco Stain), and phosphoproteins (Phospho Stain) as indicated in FIG. 2.

Accordingly, there was a substantial enrichment of 75 kDa glycoproteins and phosphoproteins in the BMF extract when compared directly with the +CL control (FIG. 2, astericks). Of the prominent proteome stained bands at 10-15 kDa and 65 kDa in the BMF extract, only the former were shared with the total cell layer controls (FIG. 2). Based upon the hypothesis that BMF are structures assembled for the specific purpose of nucleating hydroxyapatite crystals in culture and in primary bone, this comparative analysis was designed to identify proteins substantially enriched within mineralized BMF. Since mineral nucleation is a specialized function, the BMF should exhibit a specialized proteome. As anticipated, there was a clear difference (more than 5-10 fold) in 75 kDa glyco- and phosphoproteins between the BMF proteome and that of the +CL control. The absence of similar post-translationally modified proteins in the −CL control is evident (FIG. 2).

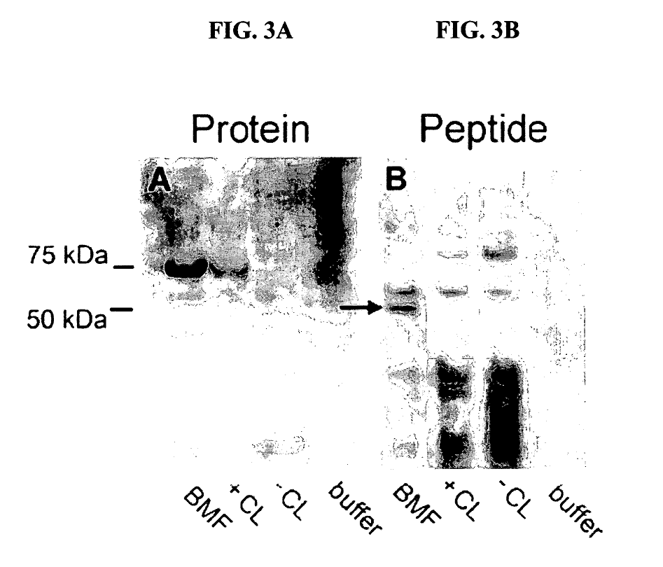

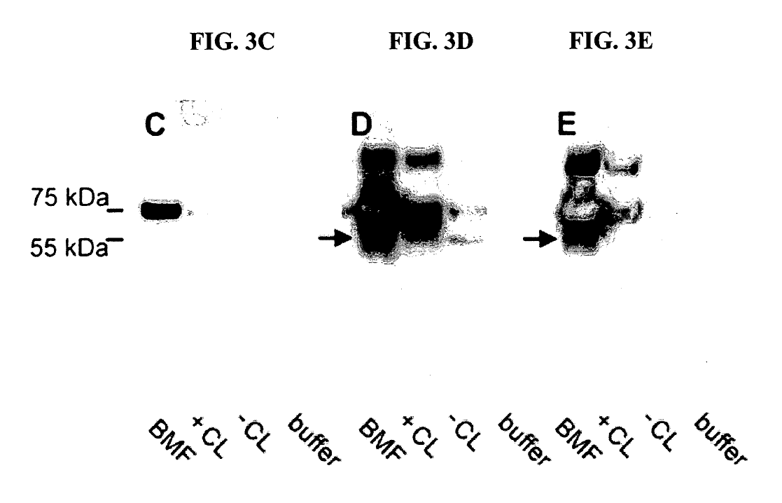

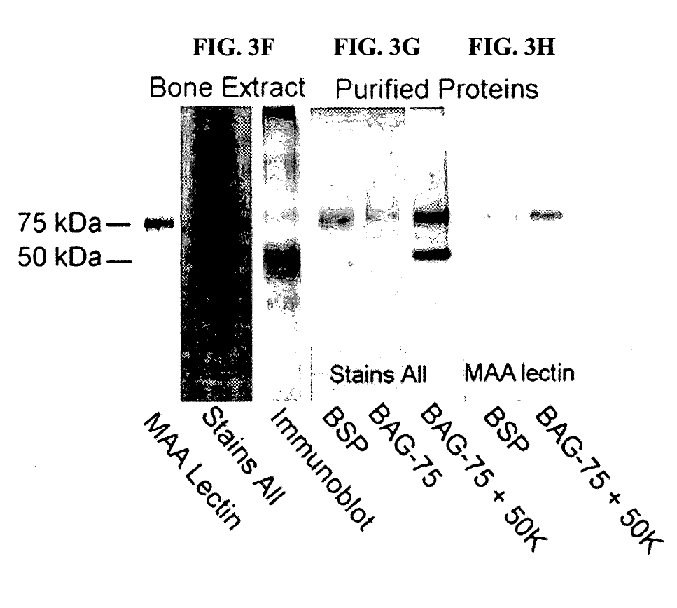

Immunoblotting studies (FIG. 3A-E) revealed that 75 kDa glycophosphoproteins BAG-75 and BSP were both dramatically enriched in BMF only in the presence of β-glycerol phosphate. Closer inspection revealed 6 BMF fractions also contained a higher relative content of BAG-75 and BSP fragments (Arrows, FIGS. 3B, D, and E). In the case of BAG-75, this was detected through the use of an N-terminal #3-13 anti-peptide antibody (#503), which preferentially recognized a 50 kDa fragment. For BSP, a 45-50 kDa fragment was observed when the full-length BSP band was purposely overloaded (FIGS. 3C, D, and E). The enrichment of full-length protein within BMF links BAG-75 and BSP with mineral nucleation, while localization of their cleavage fragments at the site of initial crystal nucleation raises a question as to whether proteolytic cleavage of BAG-75 and BSP is required for mineral nucleation within BMF.

Results with whole animals indicated that BAG-75 and BSP are the two major glycoproteins in rat bone. Specifically, total 4M guanidine HCl/0.5 M EDTA extracts of the mineralized compartment of bone contain a single 75 kDa glycoprotein band that was reactive with MAA lectin (FIG. 3F), paralleling results obtained with glycoprotein staining of UMR fractions (FIG. 2). Bone extracts, like UMR extracts, also contain a major phosphoprotein of this size revealed after Stains All staining (FIG. 3G). As shown in FIG. 3H, both purified BSP and BAG-75, but not a characteristic 50 kDa fragment of BAG-75, strongly reacted with MAA lectin. As a result, BAG-75 and BSP together comprise the 75 kDa glycophosphoprotein band whose cellular distribution specifically reflects the state of mineralization in the UMR culture model.

The proteome of the bone mineralization process is enriched with glycoproteins and phosphoproteins. Specifically, the proteome is highly enriched with both 75 kDa and 45-50 kDa forms of BAG-75 and BSP proteins. The level of the 50 kDa fragment of BAG-75 increased 2.5 fold or greater in the blood of animals experiencing new bone formation. High levels of the 75 kDa BAG-75 and BSP proteins and their respective 45-50 kDa fragments are an indication of active bone mineralization, and therefore, are strong bone marker candidates. Restricted expression of the 50 kDa BAG-75 fragment to forming bone and to bone modeling sites suggested that a 50 kDa protein assay could provide a means to detect specific changes at these sites throughout the bone formation process. Current bone formation markers do not share this specificity or a potential one-to-one relationship with the actual process of bone mineralization.

### Example 4

### Serine Protease Inhibitor AEBSF Specifically Inhibits Mineral Nucleation Without Harming the Cells

While the enrichment of BAG-75 and BSP proteins was associated with the bone mineralization process, their involvement remains elusive. The localization of their cleavage fragments to the site of initial crystal nucleation raises a question as to whether proteolytic cleavage of BAG-75 and BSP is required for mineral nucleation within BMF.

To investigate the nature of the protease activity responsible for BAG-75/BSP cleavage and the relationship of cleavage with mineralization, a variety of protease inhibitors (Table 1) were tested in the UMR model. Individual inhibitors were added to confluent cultures at 64 hours after plating and the amount of hydroxyapatite deposited within BMF was quantitated 24 hours later. In general, UMR cultures are not competent to mineralize until 60 hours after plating, reflecting an osteogenic differentiation process which leads to the production of spherical pre-BMF structures.

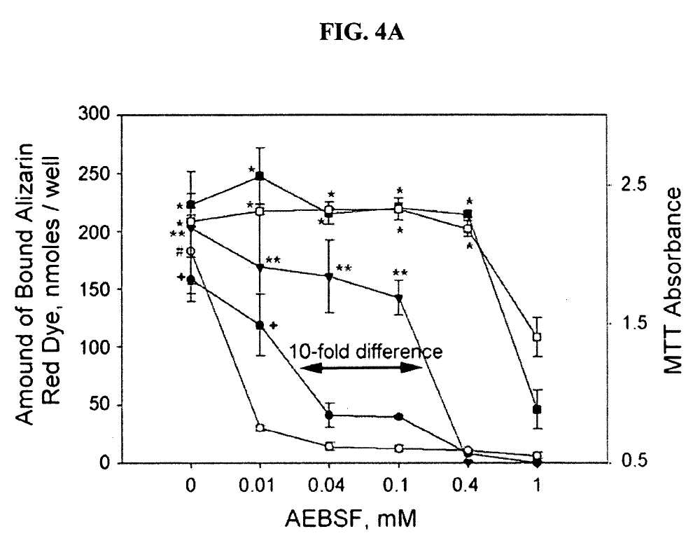

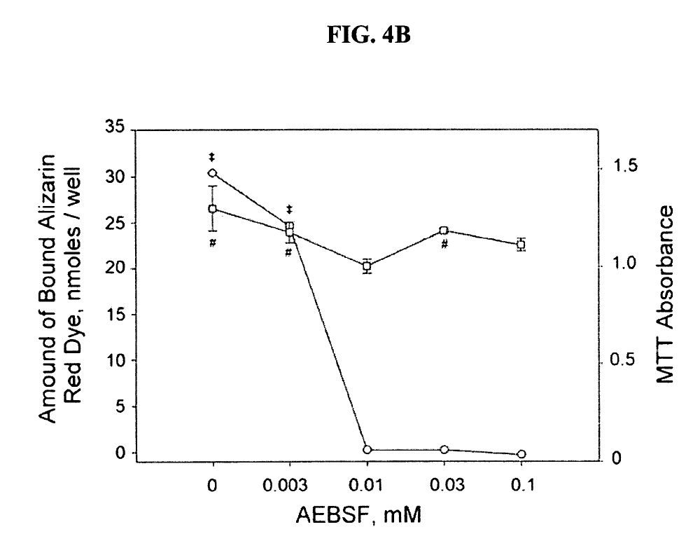

Only one inhibitor, AEBSF, blocked mineral nucleation in BMF (Table 1 and FIG. 4). AEBSF is a covalent serine protease inhibitor and was capable of completely blocking mineral nucleation at concentrations as low as 0.04 mM. None of the other protease inhibitors tested, which included inhibitors of thrombin, plasmin, plasminogen activator, and matrix metalloproteinases, diminished mineralization in the UMR system when used at their optimal recommended dosage (Table 1). When added at 64 hours after plating, AEBSF was similarly effective regardless of whether serum was included in the culture media or not (FIG. 4), demonstrating the source of the mineralization-related, AEBSF-sensitive protease was the UMR 106 cells themselves. Interestingly, the time at which AEBSF was added dramatically influenced the outcome. Assuming a baseline mineral level of 150-170 nmoles/well, the inhibitor was 10-fold less effective if present during the period in which the cells are actively proliferating and differentiating (44-64 hours after plating) rather than during the mineralization period (64-88 h after plating) (FIG. 4A).

In view of the toxicity of some protease inhibitors, the apparent inhibition of mineralization observed with AEBSF may have been a non-specific effect on cell viability, e.g., dying or dead cells may not nucleate mineral with BMF. To examine this possibility, viability of AEBSF-treated and non-treated control cultures was analyzed using the MTT assay for vital mitochondria. As shown in FIG. 4B, AEBSF was only toxic at concentrations above 0.4 mM. This effect was similar whether cells were grown in serum-sufficient or serum-replete conditions. Specifically, 0.01 to 0.1 mM AEBSF was able to block mineralization almost completely in primary calvarial cultures with little change in viable cell number when scored on day 12 (FIG. 4B). Importantly, no loss of cell viability was noted below 1 mM AEBSF, demonstrating that, in this range, the loss of mineral nucleation capacity was not due to toxicity.

Preventing the cleavage of BAG-75 and BSP completely blocks bone mineralization indicating that the cleavage event is required for the bone mineralization process. Furthermore, the protease responsible for cleavage is serine specific. Regulating the serine-protease would likely provide a means to regulate the bone mineralization process. Furthermore, while the BAG-75 and BSP full length proteins are candidates for bone mineralization markers, the fragments of BAG-75 and BSP are specific markers of actively occurring bone mineralization.

TABLE 1
Protease inhibitors tested for their effect on BMF in UMR-106 cells.

| | | | Inhibition of |
|---|---|---|---|
| | | Range of Conc. | Mineral |
| Inhibitor | Target Protease(s) | Tested | Deposition |
| AEBSF | trypsin, chymotrypsin, plasmin, thrombin, | 0.01-1 mM | Yes |
| | kallikrein, proprotein convertases | | |
| Aprotinin | trypsin, chymotrypsin and plasmin | 0-3 μg/ml | No |
| Antipain | papain and trypsin, plasmin | 100 μM | No |
| C1s inhibitor | activated complement protein C1s | 0.1-100 μg/ml | No |
| E-64 | cysteine proteases | 10 μM | No |
| Elastatinal | elastase and elastase-like proteases | 100 μM | No |
| GM 6001 | matrix metalloproteinases 2, 3, 8, and 9 | 10 μM | No |
| Hirudin | thrombin | 0.5-10 ATU | No |
| Leupeptin | trypsin-like proteases and some cysteine proteases | 100 μM | No |
| Pefabloc PL | plasmin and plasma kallikrein | 1 μM-100 μM | No |
| Pefabloc uPA | urokinase plasminogen activator | 1 μM-100 μM | No |

### Example 5

### Enrichment of a 75 kDa Phosphoglycoprotein Band in the Cell Layers of Mineralizing Cultures is Blocked by AEBSF

The mechanism involved in the requirement for BAG-75 and BSP in mineralization is unknown. According to the above-described results, the mechanism involves a serine specific protease that can be blocked by AEBSF. The mechanism and involvement of blocking the serine protease was further analyzed.

The effect of AEBSF on the protein distribution within mineralizing cultures was analyzed using cells grown until 64 hours, at which time they were fed with one of four different media conditions. The four conditions were: 1) 7 mM β-glycerol phosphate only (+BGP); 2) 7 mM β-glycerol phosphate and 0.1 mM AEBSF (+BGP+AEBSF); 3) the absence of both β-glycerol phosphate and AEBSF (−BGP), and 4) 0.1 mM AEBSF only (−BGP+AEBSF). Cell layer extracts and media fractions from all four conditions were then compared using 1-dimensional SDS-PAGE followed by staining or immunoblotting.

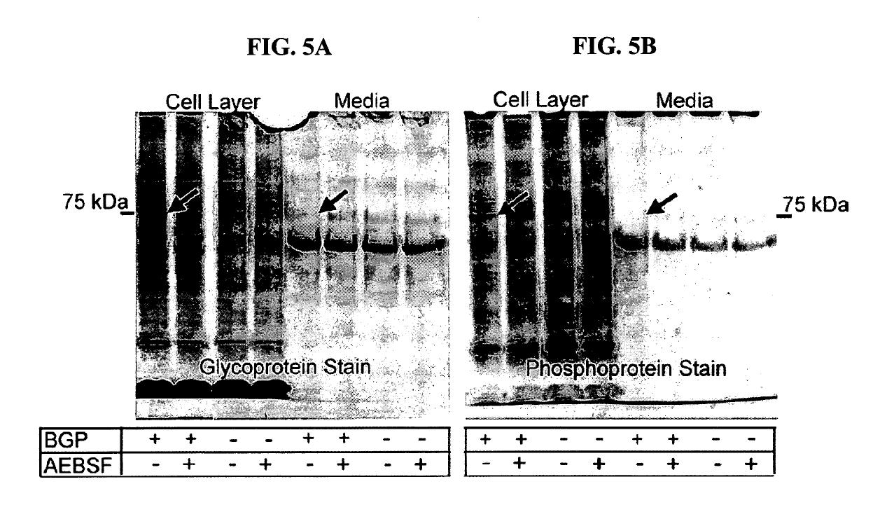

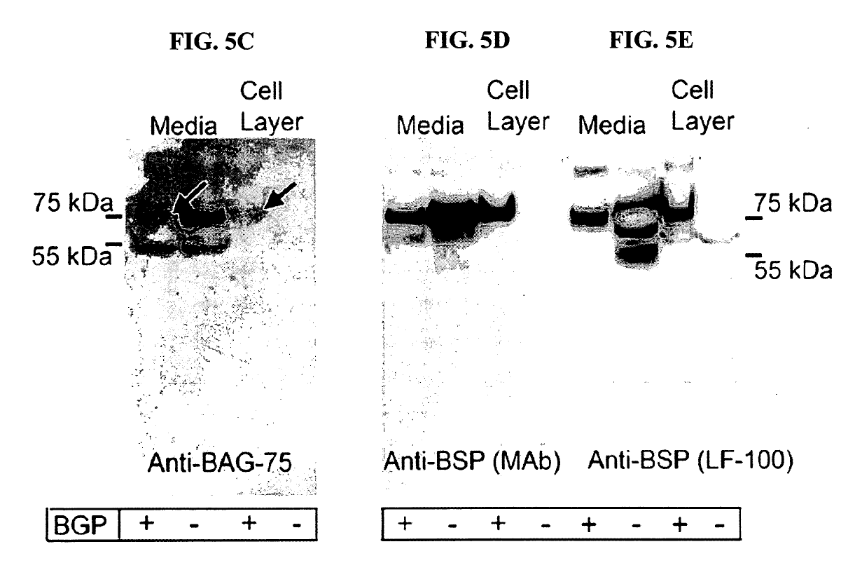

Following mineralization, the cell layer was extracted with either a 50 mM EDTA-CHAPS detergent based solution or with an 8 M urea-0.5% SDS-50 mM EDTA extraction solution as described herein. The solubility was subsequently defined by ultracentrifugation at 108,000×g for 1 h. Gel electrophoresis revealed that the 75 kDa glyco- and phosphoprotein band was lost rather specifically from the media fraction during the 24 h mineralization period (arrows, FIG. 5A).

The 75 kDa glycoprotein band was likely composed of BAG-75 and BSP because they were the only two proteins of this molecular weight in total bone extracts that react with digoxygenin labeled MAA lectin (FIG. 3H). The 75 kDa phosphoprotein band is presumed to be predominantly composed of BAG-75 since BSP from bone exhibits low phosphate content while BAG-75 contains about 44 phosphates/mole. Loss from the media fraction only occured when mineralization was occurring, not when it was blocked by inclusion of AEBSF or when β-glycerol phosphate was omitted (FIG. 5A). While similar analyses of the cell layer demonstrated that a 75 kDa glycoprotein was taken up only during mineralization progress, a comparable increase in phosphoprotein (e.g., BAG-75) staining was not observed (arrows, FIG. 5A). These conclusions were confirmed when similar 1-step extracts were probed with monospecific antibodies (FIG. 5B). While approximately one-half of the BSP was lost from the media fraction during mineralization (+BGP), a comparable amount of BSP became associated with the cell layer. Although BAG-75 protein was also lost from the media fraction only when mineralization occurred (media, +BGP), its recovery in the cell layer fraction was lower than expected. This is contrary to the known presence of BAG-75 antigen in BMF complexes prior to and during their mineralization in osteoblastic cell cultures. The use of the one-step extraction method resulted in a substantial, unexplained loss of BAG-75.

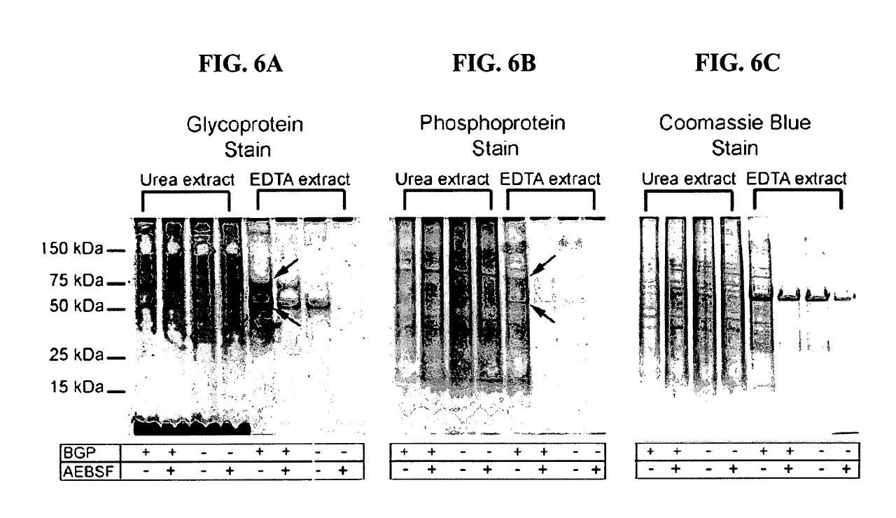

As an alternative, a two-step sequential extraction protocol was used in which the cell layer was extracted first with EDTA and then separately with 8 M urea, two of the major dissociative agents in the single-step extraction solution. To dissolve mineral crystals and release bound proteins, the cell layer was first extracted for 2 h at 4° C. with 0.05 M EDTA, pH 7.8. The EDTA extract was removed and the flasks were then treated vigorously with 8 M urea and 2% CHAPS, pH 7.8, in order to solubilize remaining proteins from the cell layer. Each extract was processed separately, subjected to 1-D SDS-PAGE, and the gels were stained with Coomassie blue dye, for glycoproteins, or for phosphoproteins. Urea-CHAPS extracts showed few differences among the four different conditions (FIG. 6). In contrast, EDTA extracts of cell layers grown only in the presence of β-glycerol phosphate displayed dramatically increased glycoprotein and phosphoprotein stained bands at 50 kDa and 75 kDa molecular weights when compared directly to that of cultures grown in the other three conditions (FIG. 6). Interestingly, general protein staining with Coomassie blue yielded a comparable pattern for all culture conditions suggesting an absence of large-scale proteolysis accompanying mineral nucleation within BMF (FIG. 6). Taken together, these findings indicated that the two-step extraction method improved recoveries of unaccounted for 75 kDa and 50 kDa glycophosphoproteins from the cell layer of mineralized cultures. Furthermore, one or more 75 kDa glycophosphoproteins present in the serum-free media cultures were specifically taken up by the cell layer (+BGP) during the mineralization period (64-88 hours) (FIGS. 5 and 6). Since LCM-captured BMF are highly enriched in a similar glycophosphoprotein band of 75 kDa (FIG. 2), the source of the latter band was the media fraction. When mineralization was blocked with AEBSF, the 75 kDa glycophosphoprotein band remained in the media fraction (FIG. 6). Likewise, in the absence of β-glycerol phosphate, the 75 kDa band remained in the media compartment (−BGP and −8 BGP+AEBSF) (FIG. 6).

Cells undergoing the mineralization process took up BAG-75 and BSP proteins residing in the media, or microenvironment. This uptake, as well as the mineralization process, was blocked with the serine protease inhibitor AEBSF. These results underline the requirement of BAG-75 and BSP uptake by cells for mineralization to occur.

### Example 6

### A Media Component is Required to Nucleate Mineral Crystals Within BMF

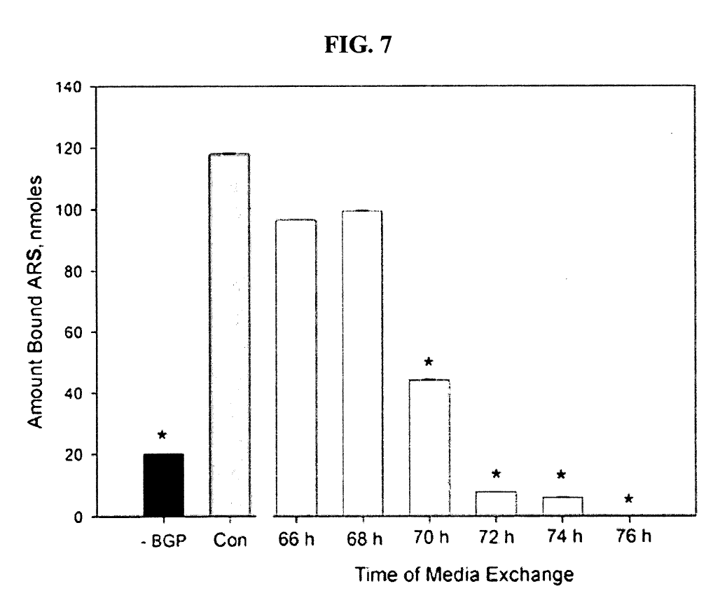

The active process of bone mineralization is dynamic, requiring factors readily available in the microenvironment. Under conditions of serum depletion, the media fraction contains exogenous BSA and proteins secreted by UMR cells. Assuming that the rate of protein secretion into the media compartment is constant over the period from 64 to 88 hours, the amount of 75 kDa glycophosphoprotein would be expected to be in the media fraction until mineral nucleation begins. Typically, the first mineral crystals appear at or near 76 hours in the UMR model. Since mineralization in UMR cultures was accompanied by the transfer of a 75 kDa glycophosphoprotein band from the media to BMF in the cell layer fraction, the amount of cell-derived media proteins could influence the amount of hydroxyapatite crystals produced. To test this, fresh media containing β-glycerol phosphate was added as usual to UMR cells at 64 hours; however, at different times over the next 24 hours, conditioned media from these flasks was again exchanged with fresh media. The amount of hydroxyapatite crystals produced per flask was determined at 88 hours by quantitating the amount of bound Alizarin red S dye. It is apparent that the effect of media swapping was more dramatic the later the exchange occurred. No significant change in mineral nucleation was observed until 4 hours or later, with the 6, 8, and 10 hour time points producing only background levels of hydroxyapatite (FIG. 7). Interestingly, if 12 hour conditioned media was removed from one flask and exchanged immediately for that in an identical culture, no change in mineral content was noted. The latter finding showed that the act of media exchange per se is not detrimental to mineralization. Thus, these results indicated for the first time that one or more media components are required for mineralization of BMF (FIG. 7).

### Example 7

### BAG-75 and BSP are Two Components which Comprise the 75 kDa Glycoprotein Band

The fact that the extracellular matrix of bone contains only two prominent glycoproteins facilitated further identification of the 75 kDa glycoprotein band participating in mineral nucleation. Specifically, total extracts of the mineralized compartment of bone matrix (G/E extracts) contained a single band at 75 kDa which reacted with MAA lectin (FIG. 3F). This result paralleled that obtained earlier with glycoprotein staining (FIG. 2). Furthermore, bone extracts, like UMR extracts, also contained a major phosphoprotein of this size revealed after Stains All staining (FIG. 3F). Finally, as shown in FIG. 3H, both purified BSP and BAG-75, but not a characteristic 50 kDa fragment of BAG-75, strongly reacted with MAA lectin. Resultantly, BAG-75 and BSP together comprise the 75 kDa glycoprotein band whose cellular distribution specifically reflected the state of mineralization in the UMR culture model.

### Example 8

### AEBSF Inhibits Proteolytic Cleavage of BAG-75 and BSP Accompanying Mineralization

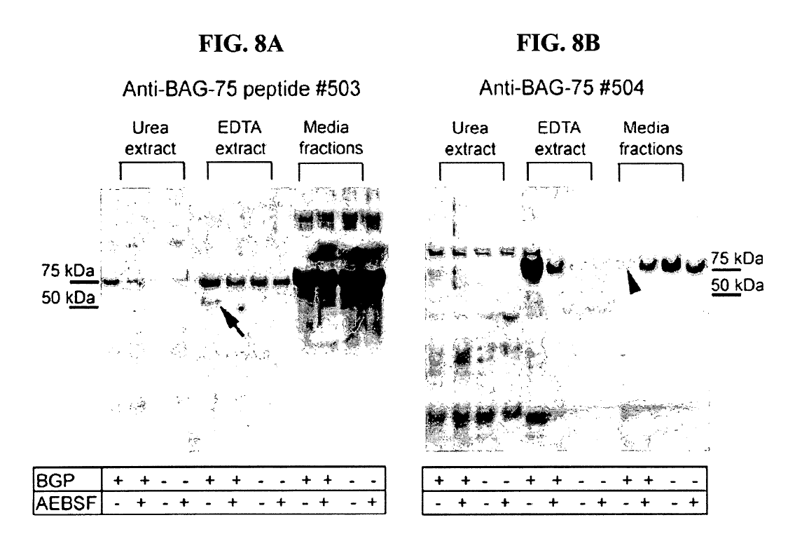

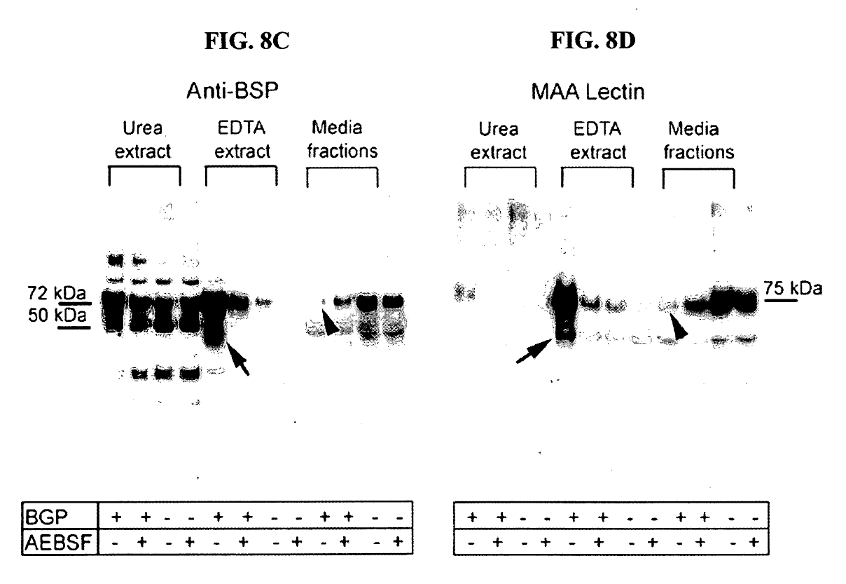

In view of the identification of BSP and BAG-75 as 75 kDa glycoproteins involved in mineral nucleation, and, the enrichment of 45-50 kDa fragments within LCM-captured BMF (FIG. 3), it was of interest to establish whether their cleavage was susceptible to AEBSF inhibition. UMR cultures were grown in the presence or absence of AEBSF and of β-glycerol phosphate. Resultant cell layer fractions were extracted with the two stage extraction protocol of 0.05 M EDTA followed by 8 M urea-2% CHAPS as described herein. For comparison, all media and cell layer fractions were electrophoresed in adjacent lanes and blotted with either MAA lectin, antibody 503 (recognizes N-terminal residues #3-13 of BAG-75), antibody 504 (recognizes BAG-75 protein), or anti-BSP antibodies (FIG. 8A-D). Consideration of these blots revealed several interesting points. First, full-length BAG-75 and BSP were taken up by the cell layer only during mineralization within BMF (FIGS. 8B and C). Second, 45-50 kDa fragments of BAG-75 (FIG. 8A) and BSP (FIG. 8C) were detected in the cell layer only when mineralization occurred. Importantly, cleavage, blocked by AEBSF, was concomitant with the inhibition of mineralization. Third, MAA lectin, which recognizes both BSP and BAG-75 (FIG. 3H), also recognizes 45-50 and 75 kDa forms in mineralized cell layer fractions (FIG. 8D). Finally, direct analyses of LCM-captured BMF have shown that the 75 kDa and 45-50 kDa fragment forms of BAG-75 and of BSP were both predominantly localized to BMF complexes (FIG. 3). In summary, AEBSF blocks uptake and cleavage of BAG-75 and BSP, as well as mineralization within BMF. In view of the known affinity of BSP and BAG-75 for hydroxyapatite crystals, it is likely that some of the uptake by the +BGP cell layer is due to direct binding to mineral. However, a major portion of these proteins taken up by the +BGP cell layer also occurs in the absence of mineral and of cleavage (+BGP+AEBSF) (FIGS. 8B and C). In this context, 86 genes have been identified that were induced within 12 hours after the addition of β-glycerol phosphate to the cultures. Control blots developed with MAA lectin confirmed earlier glycoprotein staining results showing a redistribution of a 75 kDa glycoprotein concomitant with mineral crystal nucleation (FIG. 8D). In this context, the amount of protein binding to mineral crystals may represent the difference between the respective 75 kDa bands for BAG-75 or BSP in +BGP versus +BGP+AEBSF lanes (FIGS. 8B and C). While the percentage of cleaved fragment relative to the full length BAG-75 and BSP in the cell layer of +BGP cultures was less than 50%, the amount of stained fragment was similar to that for uncleaved precursor proteins (FIG. 8 A and C) from +BGP+AEBSF cultures. It is noteworthy that unmineralized cultures contained high levels of the uncleaved, full length proteins in the media (FIG. 8A-D).

Mineralization occurs concomitantly with the uptake and cleavage of BAG-75 and BSP. Blockage of this cleavage by AEBSF led to complete inhibition of mineral nucleation within BMF.

### Example 9

### 2-D SDS-PAGE Reveals AEBSF Blocks the Cleavage and Uptake of Other Mineralization Related Proteins into the Cell Layer

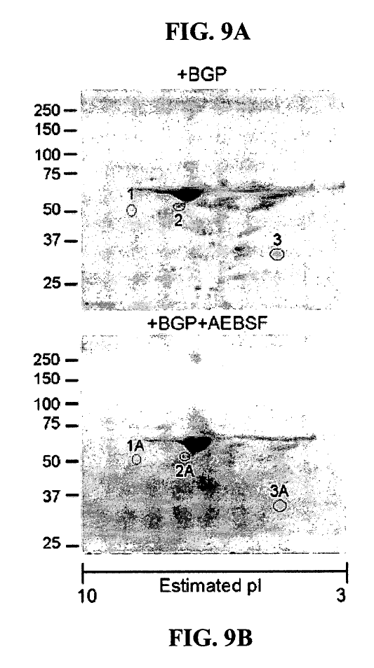

AEBSF is a general serine protease inhibitor and the inhibition of mineralization by AEBSF could very well be due to the blockage of cleavage events of other necessary proteins. To identify other proteins affected by AEBSF protease inhibition, cells were grown under serum-depleted conditions and the resultant cell layer fractions were extracted with the two-step protocol using 0.05 M EDTA and then 8 M urea-2% CHAPS. Preparations from each cell layer extract and media fraction were first isoelectro-focused on pH 3-10 IEF focusing strips. The second dimension was run on 10.5-14% Criterion slab gels. Gels were stained with colloidal Coomassie blue and aligned using the PD-Quest program to identify differences in the staining patterns for the +BGP condition compared with that for the +BGP+AEBSF condition (FIG. 9). The results depicted were representative of duplicate analyses of each condition, which were carried out on two separate preparations of each. There were no major differences detected between the two growth conditions for either the urea extract or the media fraction. However, the differences detected between the two EDTA extracts were visually dramatic (FIG. 9). Gel spots were then selected for mass spectral peptide mapping and MS/MS identification if there was at least a 2-fold difference in staining intensity between the two culture conditions. Gel plugs were excised and processed for trypsin digestion and mass spectroscopic identification as described herein leading to the assignment of over 50 protein spots in EDTA fractions from AEBSF treated and untreated control cultures. Application of the following criteria to this list identified three additional AEBSF sensitive cleavages among EDTA extractable proteins. The criteria included the following: 1) spot present in EDTA extract was absent in urea extract and in media fraction; 2) spot exhibited substantially higher staining intensity in the +BGP condition as compared with that in +BGP+AEBSF condition; 3) size of protein based on second dimension SDS-PAGE is smaller than expected; and 4) apparent isoelectric point is inconsistent with that expected for full length protein. Table 2 provides a summary list of five proteins whose cleavage was blocked by treatment with AEBSF. These proteins are procollagen C proteinase enhancer (Pcolce) protein, bone sialoprotein, 1,25-vitamin D3 membrane-associated rapid response steroid binding protein, nascent polypeptide associated complex alpha chain, and bone acidic glycoprotein-75. Evidence for cleavage for these proteins appeared consistent with prior data. This prior data includes the finding that Pcolce enhances the C-terminal propeptidase activity of BMP-1 (activin), which is required for collagen assembly, and an active fragment of Pcolce was previously identified in 3T6 fibroblast cells. N-terminal fragments of BSP have been shown to nucleate mineral crystals in vitro. Also, the amount of a 50 kDa fragment of BAG-75 in serum correlates with bone formation in a rat ovariectomy model. However, currently, there is no evidence for proteolytic activation or inactivation of 1,25-MARRS, but the data described herein suggested that such a requirement may exist.

AEBSF inhibited the cleavage of three additional proteins likely involved in bone mineralization.

TABLE 2

Summary of proteins blocked by AEBSF.
Summary of proteins in EDTA extract whose fragmentation is blocked by AEBSF

| Spot | Protein | Observed | Apparent | Expected | Method(s) for | Peptides identified |
|---|---|---|---|---|---|---|
| # | Identification | MW (Da) | MW (Da) | pI | identification | (Mascot score) |
| 1 | Procollagen C | 45,000 | 53,835 | 8.5 | Differential | FDVEPDTYCR (59) |
| | proteinase | | | | staining after | TGDLDLPSPASGTSLK (49) |
| | enhancer protein | | | | 2-D SDS- | SGTLQSNFCSSSLVVTGTVK (75) |
| | | | | | PAGE and | |
| | | | | | mass | |
| | | | | | spectroscopy | |
| 2 | 1,25-D3-MARRS | 45,000 | 57,079 | 5.4 | Differential | LNFAVASR (63) |
| | (ERp57) | | | | staining after | YGVSGYPTLK (65) |
| | | | | | 2-D SDS- | LAPEYEAAATR (88) |
| | | | | | PAGE and | FAHTNVFSLVK (74) |
| | | | | | mass | DLFSDGHSEFLK (72) |
| | | | | | spectroscopy | |
| 3 | Nascent | 30,000 | 221,512 | 9.4 | Differential | DIELVMSQANVSR (75) |
| | polypeptide | | | | staining of 2-D | SPASDTYIVFGEAK (79) |
| | associated | | | | SDS after | NILFVITKPDVYK (80) |
| | complex, alpha | | | | PAGE and | |
| | chain | | | | mass | |
| | | | | | spectroscopy | |
| 4 | Bone acidic | 50,000 | 75-80,000 | 4.5-5.0 | 1-D SDS- | N/A |
| | glycoprotein-75 | | | | PAGE | |
| | | | | | immunoblottin | |
| | | | | | g | |
| 5 | Bone sialoprotein | 45-50,000 | 75-80,000 | 6.0 | 1-D SDS- | N/A |
| | | | | | PAGE | |
| | | | | | immunoblottin | |
| | | | | | g] | |

### Example 10

### SKI-1 Serine Protease is Present in Biomineralization Foci

A soluble, 98 kDa active form of SKI-1 is expressed during active mineralization. SKI-1 is a serine protease that is sensitive to inhibition by AEBSF. As serine proteases play a criticalrole in the activation and regulation of a number of biological processes, SKI-1 is a candidate activator of the fragmentation of proteins involved in bone mineralization. Such proteins include BAG-75 and BSP. Although it is not known whether SKI-1 sites are present in BAG-75 and account for its fragmentation during mineralization, there are several SKI-1 candidate cleavage sequences present in BSP, 1,25-D3-MARRS receptor, and Pcolce.

To determine if SKI-1 expression correlates with the bone mineralization process, SKI-1 expression was compared among BMFs, mineralizing cell cultures, and un-mineralizing cell cultures. Specifically, total cell extracts from phosphate supplemented and control cultures were compared with that for the laser capture microscope purified BMF. UMR cells were cultured on glass slides and the resultant biomineralization foci were purified using laser capture microscopy. As controls, the total cell layer fraction from a mineralized culture (+CL) and an un-mineralized culture (−CL) were extracted separately and subjected to SDS-PAGE and immunoblotting along with laser captured BMF or buffer alone. Identical amounts of protein were loaded into each lane and the resultant blot was probed with anti-SKI-1 antibodies.

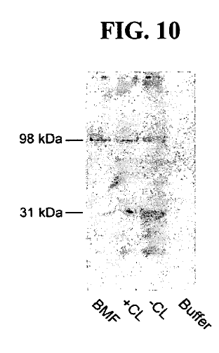

SKI-1 was present in all mineralizing samples as a 98 kDa soluble enzyme (FIG. 10, +CL and BMF); the predominant forms in non-mineralizing cultures were smaller than 35 kDa (FIG. 10, −CL). Under normal conditions SKI-1 resides in the cis/medial Golgi as a ˜106 kDa active transmembrane form. In specific circumstances SKI-1 is transported to the plasma membrane, and auto-catalytically shed as a ˜98 kDa catalytically active, soluble enzyme. The amount of 98 kDa form detected is similar in the total culture extracts and in the purified BMF preparation. While not indicative of a quantitative enrichment of SKI-1 to BMF complexes, the results demonstrate the association of SKI-1 with structures mediating initial mineral nucleation.

### Example 11

### Detection of Bone Mineralization Using Human Serum Samples

The requirement of BAG-75 cleavage for bone mineralization indicates that BAG-75 and its 50 kDa cleavage fragment are potentially strong markers of bone mineralization. The specificity of BAG-75 and its cleavage fragment to the mineralization process and their availability in the microenvironment are good characteristics for being used as detection markers in screening assays.

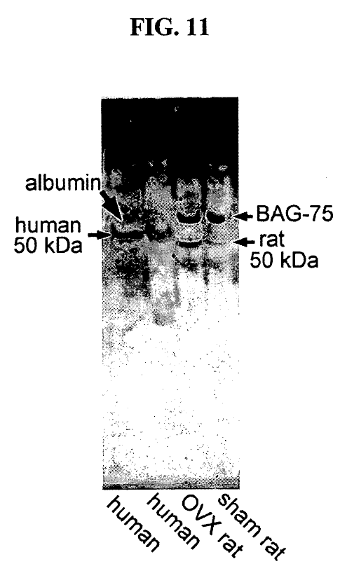

The 50 kDa fragment of BAG-75 was detected in human serum samples using anti-VARYQNTEEEE antisera (FIG. 11). Serum levels of BAG-75 and fragments thereof were detected and compared between human, ovariectomized rat (OVX), and sham-operated rat (sham) serum samples. The OVX and sham rat sera were obtained on day 21 after surgery, which is the peak of new bone formation in the OVX rat model of increased bone turnover. OVX and sham rats were obtained from Charles River, Inc. Surgeries were performed at their facility and the animals were shipped shortly thereafter. Three different dilutions of human and rat serum were run on the same 4-20% gradient SDS PAGE gel. The gel was blotted onto PVDF membrane and then subjected to chemiluminescent detection with 1/50,000 diluted anti-VARYQNTEEEE antibodies. Newly prepared anti-VARYQNTEEEE antisera were pooled from two rabbits produced against a bovine serum albumin (BSA) peptide conjugate. The antisera were adsorbed twice with BSA-Sepharose prior to use and the antibody buffer contained 0.6 mg/ml BSA to prevent reactivity with human or rat serum albumin on the blots. Bands were detected in human and rat sera at the two highest dilutions ( 1/100 and 1/500). For clarity, only the 1/100 dilution lanes are shown. The 50 kDa protein content after OVX was higher than the content in sham-operated rats (FIG. 11, small arrow); however, a 75 kDa BAG-75 band was also seen in rat sera.

Normal human serum contained an approximately 50 kDa band that reacted similarly with anti-VARYQNTEEEE antibodies as the freshly prepared OVX rat serum. The intensity of the human band was weaker than that for OVX indicating a lower concentration for human 50 kDa protein than in the rat model. Consistent with the colorimetric data presented in previous Examples, the concentration of the 50 kDa protein was higher in OVX than in sham operated rats. Under conditions of chemiluminescent detection, both the 75 kDa BAG-75 and 50 kDa bands were observed in OVX and sham rats. Chemiluminescence detection is about 50-100 times more sensitive than colorimetric detection, accounting for the difference in detection of the 75 kDa BAG-75 fragment in FIG. 10 and the previously described Examples. The human 50 kDa band (FIG. 11, small arrow) was apparently slightly larger than that for rat due to differences in cleavage or phosphorylation. The position of the human serum albumin non-reactive negative band is noted with a large arrow in FIG. 11.

In summary, human serum and rat sera contained a similar sized 50 kDa band reactive with anti-VARYQNTEEEE peptide antibodies. The results suggested that normal human serum contained a lower concentration of 50 kDa protein than OVX serum, the level for which was shown to reflect the increased (induced) amount of bone formation occurring 21 days after ovariectomy. Recognition of 75 kDa BAG-75 in rat serum by anti-VARYQNTEEEE antibodies was anticipated. Several alternative approaches to specifically remove the 75 kDa BAG-75 fragment prior to analysis for 50 kDa protein are available, including adsorption with immobilized lectins or adsorption onto hydroxyapatite beads.

Alternatively, human serum levels of immunoreactive BAG-75 fragment may be determined by the enzyme-linked immunosorbent assay (ELISA) prepared with antibody recognizing BAG-75 fragments. Microtiter plates (96 well) may be coated with BAG-75 fragment specific antibody (1 μg/ml, 100 μl per well) and stored overnight at 4° C. Purified BAG-75 fragment standards, keyhole limpet hemocyanin peptide conjugate standard (VARYQNTEEEE), or samples may be added to individual wells in a total volume of 100 μl of phosphate-buffered saline containing 0.05% Tween 20 and 0.5% gelatin (dilution buffer) and then incubated at 37° C. for 90 minutes. Biotin labeled BAG-75 fragment antibody may be added to each well at a concentration of 1 μg/ml in a total of 100 μl and incubated at 37° C. for 60 minutes. Peroxidase-avidin, at a concentration of 1 μl/ml in a total volume of 100 μl may be added and subsequently incubated at 37° C. for 30 minutes. The color reaction may be performed by adding to each well 100 μl of freshly prepared substrate solution and 0.03% H₂O₂ in 0.1 M sodium citrate (pH 4.3) and then incubating the mixture at room temperature for 30 minutes. The plates may be read at 405 nm with a plate reader to detect the presence of the BAG-75 fragment and level of bone mineralization.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the claims.

What is claimed is:

1. A method of detecting bone mineralization comprising:
a. providing a biological sample;
b. assaying the biological sample for an altered level of at least one bone mineralization marker, wherein the marker is selected from the group consisting of BAG-75, fragments of BAG-75, about a 50 kDa fragment of BAG-75, SKI-1, fragments of SKI-1, and about a 98 kDa fragment of SKI-1; and
c. correlating the altered level with an alteration in bone mineralization.

2. The method of claim 1, wherein the assaying method is a protein detection method.

3. The method of claim 1, wherein the biological sample is selected from the group consisting of serum, blood, urine, synovial fluid, saliva, tissue biopsy, surgical specimen, autopsy material, and body cells.

4. The method of claim 3, wherein the biological sample is serum.

5. The method of claim 1, wherein the altered level is elevated or reduced compared to an initial baseline reading from the same sample source.

6. The method of claim 1, wherein the altered level is elevated or reduced compared to a normal standard average.

7. The method of claim 1, wherein the altered level is at least about a 1.5 standard deviation above or below the normal mean.

8. A method of detecting at least one bone mineralization marker for monitoring bone mineralization, wherein the method comprises:
a. providing a biological sample;
b. assaying the biological sample for an altered level of at least one bone mineralization marker, wherein the marker is selected from the group consisting of BAG-75, fragments of BAG-75, about a 50 kDa fragment of BAG-75, SKI-1, fragments of SKI-1, and about a 98 kDa fragment of SKI-1; and
c. correlating the altered level with an alteration in bone mineralization.

9. The method of claim 8, wherein the assaying method is a protein detection method.

10. The method of claim 8, wherein the biological sample is selected from the group consisting of serum, blood, urine, synovial fluid, saliva, tissue biopsy, surgical specimen, autopsy material, and body cells.

11. The method of claim 10, wherein the biological sample is serum.

12. The method of claim 8, wherein the altered level is elevated or reduced compared to an initial baseline reading from the same sample source.

13. The method of claim 8, wherein the altered level is elevated or reduced compared to a normal standard average.

14. The method of claim 8, wherein the altered level is at least about a 1.5 standard deviation above or below the normal mean.

15. A method of monitoring bone mineralization comprising:
a. providing a first biological sample;
b. assaying the first biological sample for an altered level of at least one bone mineralization marker;
c. providing at least a second biological sample;
d. assaying the second biological sample for an altered level of at least one bone mineralization marker;
e. comparing the amount of bone mineralization markers from each sample; and
f. correlating the altered level with an alteration in bone mineralization.

16. The method of claim 15, wherein the bone mineralization marker is selected from the group consisting of BAG-75, fragments of BAG-75, about a 50 kDa fragment of BAG-75, SKI-1, fragments of SKI-1, about a 98 kDa fragment of SKI-1, and any combination thereof.

17. The method of claim 15, wherein the detection method is a protein detection method.

18. The method of claim 15, wherein the biological sample is selected from the group consisting of serum, blood, urine, synovial fluid, saliva, tissue biopsy, surgical specimen, autopsy material, and body cells.

19. The method of claim 18, wherein the biological sample is serum.

20. The method of claim 15, wherein a third biological sample is provided and assayed.

21. A method of using BAG-75 or fragment thereof to detect bone mineralization comprising:
a. obtaining BAG-75 or fragment thereof,
b. forming an antibody directed to BAG-75 or fragment thereof,
c. using the antibody to assay a biological sample for an altered level of BAG-75 or fragment thereof, and
d. correlating the altered level with an alteration in bone mineralization.

22. The method of claim 21, wherein the level of BAG-75 or fragment thereof is elevated or reduced compared to an initial baseline reading from the same sample source.

23. The method of claim 21, wherein the level of BAG-75 or fragment thereof is elevated or reduced compared to a normal standard average.

24. The method of claim 21, wherein the assaying method is a protein detection method.

25. The method of claim 21, wherein the biological sample is selected from the group consisting of serum, blood, urine, synovial fluid, saliva, tissue biopsy, surgical specimen, autopsy material, and body cells.

26. The method of claim 25, wherein the biological sample is serum.

27. The method of claim 21, wherein the altered level is at least about a 1.5 standard deviation above or below the normal mean.

28. A method of using SKI-1 or fragment thereof to detect bone mineralization comprising:
a. obtaining SKI-1 or fragment thereof,
b. forming an antibody directed to SKI-1 or fragment thereof,
c. using the antibody to assay a biological sample for an altered level of SKI-1 or fragment thereof, and
d. correlating the altered level with an alteration in bone mineralization.

29. The method of claim 28, wherein the level of SKI-1 or fragment thereof is elevated or reduced compared to an initial baseline reading from the same sample source.

30. The method of claim 28, wherein the level of SKI-1 or fragment thereof is elevated or reduced compared to a normal standard average.

31. The method of claim 28, wherein the assaying method is a protein detection method.

32. The method of claim 28, wherein the biological sample is selected from the group consisting of serum, blood, urine, synovial fluid, saliva, tissue biopsy, surgical specimen, autopsy material, and body cells.

33. The method of claim 32, wherein the biological sample is serum.

34. The method of claim 28, wherein the altered level is at least about a 1.5 standard deviation above or below the normal mean.

35. A kit for detecting bone mineralization comprising:
a. a container; and
b. at least one antibody, wherein the antibody specifically binds at least one bone mineralization marker.

36. The kit of claim 35, wherein the antibody specifically binds a bone mineralization marker selected from the group consisting of BAG-75, fragments of BAG-75, about a 50 kDa fragment of BAG-75, SKI-1, fragments of SKI-1, and about a 98 kDa fragment of SKI-1.

37. The kit of claim 35, wherein the antibody is labeled with a detection reagent selected from the group consisting of a radiolabel, a fluorescent tag, an enzymatic tag, a fluorogenic substrate tag, or a chromogenic substrate tag.

38. A kit for detecting bone mineralization comprising:
a. a container;
b. an instruction insert;
c. at least one antibody, wherein the antibody specifically binds at least one bone mineralization marker; and,
d. at least one standard sample, wherein a test sample may be compared to the standard sample to detect elevated or reduced levels of the bone mineralization marker.

39. The kit of claim 38, wherein the antibody specifically binds a bone mineralization marker selected from the group consisting of BAG-75, fragments of BAG-75, about a 50 kDa fragment of BAG-75, SKI-1, fragments of SKI-1, and about a 98 kDa fragment of SKI-1.

40. The kit of claim 38, wherein the antibody is labeled with a detection reagent selected from the group consisting of a radiolabel, a fluorescent tag, an enzymatic tag, a fluorogenic substrate tag, or a chromogenic substrate tag.


SEQUENCE LISTING (1 sequence)

SEQ ID NO: 1 - protein, 11 aa, Rattus rattus
VARYQNTEEEE